SEQUENCE LISTING (7 sequences)

SEQ ID NO: 1 - protein, 295 aa, Homo sapiens [sequence omitted]

SEQ ID NO: 2 - protein, 295 aa, Mus musculus
MSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTAAQPEVADWSEGVQVPSVPIQQFPTEDWSAQPATEDWSAAPTAQATEWVGATTEWS

SEQ ID NO: 3 - protein, 8 aa, Escherichia coli
DYKDDDDK

SEQ ID NO: 4 - protein, 194 aa, Homo sapiens
RFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 5 - protein, 194 aa, Mus musculus
RFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTAAQPEVADWSEGVQVPSVPIQQFPTEDWSAQPATEDWSAAPTAQATEWVGATTEWS

SEQ ID NO: 6 - RNA, 21 nt, Homo sapiens
gcucgugcaauuguugccauu

SEQ ID NO: 7 - RNA, 21 nt, Homo sapiens
ggcagugaccaaggaggaauu

(19) United States
(12) Patent Application Publication
(10) Pub. No.: US 2023/0212250 A1
(43) Pub. Date: Jul. 6, 2023

(54) A METHOD OF DECREASING CONCENTRATION OF TAU (T) PROTEIN AND/OR PHOSPHORYLATED TAU (T) PROTEIN

(71) Applicant: UNIVERSITY OF THE WITWATERSRAND, JOHANNESBURG, JOHANNESBURG (ZA)

(72) Inventors: Stefan Franz Thomas WEISS, Johannesburg (ZA); Katelyn CUTTLER, Johannesburg (ZA); Tyrone Chad OTGAAR, Johannesburg (ZA); Eloise VAN DER MERWE, Johannesburg (ZA); Monique J. BIGNOUX, Johannesburg (ZA)

(73) Assignee: UNIVERSITY OF THE WITWATERSRAND, JOHANNESBURG, JOHANNESBURG (ZA)

(21) Appl. No.: 17/609,731

(22) PCT Filed: May 11, 2020
(86) PCT No.: PCT/IB20/54443

§ 371 Date: Nov. 8, 2021

(30) Foreign Application Priority Data
May 9, 2019 (ZA) 2019/02879

(51) Int. Cl. C07K 14/47; C07K 14/705; C07K 14/78

(52) U.S. Cl. CPC C07K 14/4711; C07K 14/705; C07K 14/78

(57) ABSTRACT

This disclosure relates to a method of decreasing concentration of tau (τ) protein and/or phosphorylated tau (τ) protein in a target cell of a human or animal subject having Alzheimer's Disease (AD). The disclosure extends to use of biopharmaceutical agents including (i). 37 kDa/67 kDa laminin receptor precursor/high affinity laminin receptor (LRP/LR) and/or a fragment thereof, or (ii). a transfecting agent for the expression of LRP/LR, for use in treating Alzheimer's Disease (AD).

### REFERENCE TO SEQUENCE LISTING SUBMITTED VIA EFS-WEB

This application includes an electronically submitted sequence listing in .txt format. The .txt file contains a sequence listing entitled “U 020899-1_ST25.txt” created on Sep. 27, 2022 and is 9,460 bytes in size. The sequence listing contained in this .txt file is part of the specification and is hereby incorporated by reference herein in its entirety.

### FIELD OF DISCLOSURE

This disclosure relates to a method of decreasing concentration of tau (τ) protein and/or phosphorylated tau (τ) protein in a target cell of a human or animal subject having Alzheimer's Disease (AD). The disclosure extends to use of biopharmaceutical agents including (i). 37 kDa/67 kDa laminin receptor precursor/high affinity laminin receptor (LRP/LR) and/or a fragment thereof, or (ii). a transfecting agent for the expression of LRP/LR, for use in decreasing concentration of tau (τ) protein and/or phosphorylated tau (τ) protein in a target cell of a human or animal subject having Alzheimer's Disease (AD).

### BACKGROUND

Alzheimer's disease (AD) is the most prevalent neurodegenerative disorder resulting in dementia in the elderly. Currently, approximately 50 million people worldwide are suffering from AD. This disease is a result of two neuropathological features, extracellular amyloid plaques caused by aggregation of amyloid beta (Aβ) peptides and intracellular neurofibrillary tangles caused by accumulation of hyperphosphorylated tau (τ) protein.

The sequential cleavage of the amyloid precursor protein (APP) by beta (β) and gamma (γ) secretases results in the shedding of the 4 kDa Aβ which aggregates to form amyloid plaques. Conventionally, most AD related research has been directed toward Aβ. Tau (τ) is a microtubule-associated protein that normally functions to stabilize microtubules. Hyperphosphorylation of tau (τ) causes its dissociation from microtubules, ultimately leading to cell death. The biochemical pathways associated with Aβ and tau (τ) neuropathologies are substantially dissimilar and treatment methodologies successfully targeting the one will not necessarily be successful on the other.

AD is characterized by disruption of neuronal circuits, loss of synapses and neurotoxicity as a result of the two neuropathological agents, namely: amyloid plaques and neurofibrillary tangles. Amyloid plaques are due to an extracellular aggregation of amyloid beta (Aβ) peptides while neurofibrillary tangles form from the intracellular accumulation of hyperphosphorylated tau (τ). The extracellular amyloid plaques are neurotoxic and clump between neurons, disrupting their functions and leading to apoptosis. Meanwhile, neurofibrillary tangles accumulate in specific brain regions involved in memory and block the microtubular transport system, which harms synaptic communication. Overall, these agents are able to damage many areas of the brain which ultimately results in death. Downregulation of 37 kDa/67 kDa laminin receptor precursor/high affinity laminin receptor (LRP/LR) is provided as a solution to decrease Aβ notably in U.S. Pat. No. 9,365,647B2 which is fully incorporated herein by reference thereto.

This disclosure seeks to ameliorate the disadvantages known in the art and/or to provide a method of decreasing the concentration of tau (τ) protein and/or phosphorylated tau (τ) protein in a target cell of a human or animal subject having Alzheimer's Disease (AD).

### SUMMARY

In accordance with a first aspect of this disclosure there is provided a method of decreasing concentration of tau (τ) protein and/or phosphorylated tau (τ) protein in a target cell of a human or animal subject having Alzheimer's Disease (AD), the method comprising the following steps:
(i) transfecting the cell to produce 37 kDa/67 kDa laminin receptor precursor/high affinity laminin receptor (LRP/LR) and/or a fragment thereof by employing a transfecting agent; or
(ii) providing the cell with LRP/LR and/or fragments thereof.
such that in use, Steps (i) or (ii) provide an overexpression of LRP/LR in the target cell together with a concomitant increase in reverse transcriptase of telomerase (hTERT), which in turn results in decreasing concentration of tau (τ) protein and/or phosphorylated tau (τ) protein in the target cell.

The target cells may typically include brain cells of a human or animal having AD. The target cells may include nerve cells. The target cells may include neurons and glia. The glia may include ependymal cells, astrocytes, microglial cells, and oligodendrocytes.

The transfecting agent and/or the LRP/LR and/or the fragment of LRP/LR may be provided as a delivery means formulated as a pharmaceutical composition, which pharmaceutical composition may further include a pharmaceutical carrier for parenteral or non-parenteral administration to the subject. The delivery means may be adapted to facilitate site specific delivery. The delivery means may be included into, dispersed, or surrounded by the pharmaceutical carrier to form a pharmaceutical composition. The pharmaceutical carrier may be for parenteral or non-parenteral administration to the subject. The delivery means may be adapted to facilitate site specific delivery. The pharmaceutical carrier may be adapted to facilitate site specific delivery.

Non-parenteral administration may include at least one of, but not limited to, the following group: oral, nasal, rectal, vaginal, optical and transdermal administration. Typically, non-parenteral administration may be oral. Parenteral administration may include at least one of intravenous, subcutaneous and intramuscular administration. Typically, parenteral administration may be intravenous.

The transfecting agent may be pCIneo-moLRP::FLAG plasmid.

LRP/LR may comprise a peptide/protein sequence listing as set forth in SEQ ID NO: 1 or SEQ ID NO: 2, or a fragment thereof.

SEQ ID NO: 1 may be a peptide/protein sequence for human LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTATQPEV
ADWSEGVQVPSVPIQQFPTEDWSAQPATEDWSAAPTAQATEWVGATTDW
S

SEQ ID NO: 2 may be a peptide/protein sequence for mouse (Mus musculus) LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTAAQPEV
ADWSEGVQVPSVPIQQFPTEDWSAQPATEDWSAAPTAQATEWVGATTEW
S

It is to be understood that LRP/LR is highly conserved and homologs or fragments of SEQ ID NO: 1 and SEQ ID NO: 2, and/or homologs of the fragments may also utilized in order to exercise the invention described, illustrated and/or exemplified herein.

LRP/LR may comprise a peptide/protein sequence listing having at least 80% homology to the sequences as set forth in SEQ ID NO: 1 or SEQ ID NO: 2, or a fragment thereof.

LRP/LR may comprise homologs or fragments thereof, and homologs of the fragments, wherein LRP/LR may comprise a peptide/protein sequence listing as set forth in SEQ ID NO: 1 or SEQ ID NO: 2.

The peptide/protein sequence of LRP/LR or a homolog or fragment thereof, or a homolog of the fragment, may be bound to, or bonded with, or joined to, or conjugated with, or associated with, an additional protein sequence, amino acid sequence, peptide, protein, or antibody. Alternatively, and/or additionally, the peptide/protein sequence of LRP/LR may form part of a larger and/or longer peptide/protein sequence. In a certain embodiment of the invention LRP/LR may be bound to, or bonded with, or joined to, or conjugated with, or associated with, FLAG protein, such that in use, the LRP/LR may be tagged with FLAG. FLAG protein may include a peptide/protein sequence that includes at least a sequence motif DYKDDDDK (SEQ ID NO:3).

It is to be understood that the step of transfecting the cell to produce 37 kDa/67 kDa laminin receptor precursor/high affinity laminin receptor (LRP/LR) and/or a fragment may take place via known procedures in the art, including introduction into the cell of the transfecting agent. The step of transfecting the cell may upregulate LRP/LR to cause overexpression of LRP/LR.

An example embodiment of a fragment of the peptide/protein sequence listing is exemplified as SEQ ID NO: 4 corresponding to a fragment of SEQ ID NO:1 from 102 to 295 and/or SEQ ID NO:5 corresponding to a fragment of SEQ ID NO: 2 from 102 to 295.

SEQ ID NO: 4 may be a peptide/protein sequence for a fragment of human LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTATQPEVADWSEGVQVPSVPIQQFPTEDW
SAQPATEDWSAAPTAQATEWVGATTDWS

SEQ ID NO: 5 may be a peptide/protein sequence for a fragment of mouse LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTAAQPEVADWSEGVQVPSVPIQQFPTEDW
SAQPATEDWSAAPTAQATEWVGATTEWS.

The method extends generally to upregulation of LRP/LR expression in the target cells.

The method of the first aspect of this disclosure may include a Step (iii), wherein Step (iii) includes:
(iii) continuing and/or repeating Steps (i) and/or Step (ii) for a first period of time in human or animal subject having Alzheimer's Disease (AD).

The Step (iii) may take place weekly for the first period of time. The Step (iii) may take place daily for the first period of time. The Step (ii) may take place daily, weekly, monthly or any period of time in between and including the aforementioned. The first period of time may be at least six (6) months. The first period of time may be at least 12 months.

The method of the first aspect of this disclosure may include a Step (iv), wherein Step (iv) includes:
(iv) down regulation of LPR/LR prior to any one of Steps (i). (ii) and (iii) being completed, alternatively after any of Steps (i), (ii) and (iii) being completed, further alternatively together with any one of Steps (i), (ii), and (iii).

The Step (iv) provides for combination treatment of Alzheimer's Disease by providing the human or animal subject anti-37 kDa/67 kDa laminin receptor (LRP/LR) specific antibody (or fragment thereof) such that binding occurs between a surface epitope of 37 kDa/67 Kda laminin receptor (LRP/LR) and the anti-37 kDa/67 kDa laminin receptor (LRP/LR) specific antibody (or fragment thereof) causing a decrease in the concentration of amyloid beta (Aβ) peptide in the human or animal.

Step (iv) may alternatively, or additionally, include providing to the human or animal subject a nucleotide sequence, preferably shRNA of SEQ ID NO: 6 and/or SEQ ID NO: 7, such that binding occurs between mRNA of 37 kDa/67 kDa laminin receptor (LRP/IR) and the shRNA causing downregulation of the 37 kDa/67 kDa laminin receptor (LRP/LR) which in turn causes a decrease in the concentration of amyloid beta (Aβ) peptide in the human or animal.

SEQ ID NO: 6 may be gcucgugcaa uuguugccau u. The sequence may be for Homo sapiens RNA.

SEQ ID NO: 7 may be ggcagugacc aaggaggaau u. The sequence may be for Homo sapiens RNA.

Step (iv) downregulates LRP/LR which causes a decrease in Aβ. The reduction in Aβ shedding may be a reduction relative to Aβ shedding in a normal healthy human or animal, or it may be a reduction relative to Aβ shedding in a human or animal suffering from AD.

Step (iv) is counterintuitive to provide before, during or after any one or all of Steps (i), (ii) and (iii). Steps (i) to (iii) upregulate LRP/LR causing a decrease in concentration of tau (τ) protein and/or phosphorylated tau (τ) protein. It is known that the biochemical pathways associated with Aβ and tau (τ) neuropathologies are substantially dissimilar and treatment methodologies successfully targeting the one will not necessarily be successful on the other.

Concomitant and/or sequential treatment of the Aβ and tau (τ) neuropathologies via the upregulation and downregulation of LRP/LR as described herein provides a surprising and unexpected solution to ameliorating disadvantages of both the Aβ and tau (τ) neuropathologies. The skilled person would not have considered upregulation of LRP/LR to ameliorate Alzheimer's Disease (AD), and would certainly not have considered a combination therapy including the concomitant and/or sequential upregulation and downregulation of LRP/LR.

It is to be understood in regard to the downregulation using an anti-37 kDa/67 kDa laminin receptor (LRP/LR) specific antibody, that the binding between the surface epitope of 37 kDa/67 kDa laminin receptor precursor/high affinity laminin receptor (LRP/LR) (or a fragment thereof) and antibody causes a reduction in Aβ shedding.

The anti-37 kDa/67 kDa laminin receptor (LRP/LR) specific antibody, or fragment thereof, may be raised against the cell surface protein being 37 kDa/67 kDa laminin receptor precursor/high affinity laminin receptor (LRP/LR). In a preferred embodiment the antibody is raised against LRP/LR or against a protein having 80% or greater 20 homology with LRP/LR. The antibody, or fragment thereof, may be a F(ab′)2 fragment, a Fab fragment scFv, a bi-specific scFv, a tri-specific scFv, a single chain or tandem diabody, a single domain antibody (dAb), a minibody or a molecular recognition unit (MRU). Furthermore, the antibody, or fragment thereof, may be monovalent, bivalent or multivalent. The antibody, or fragment thereof, may additionally comprise at least one further antigen-interaction site and/or at least one further effector domain. In a preferred embodiment of the invention, the antibody or fragment thereof may be an anti-laminin receptor specific antibody, preferably an anti-LRP/LR specific antibody, further preferably IgG1-iS18.

In a preferred embodiment of the disclosure, the method for reducing Aβ shedding caused by the proteolytic cleavage of APP by beta (P) and gamma (γ) secretases, the method comprises contacting LRP/LR with IgG1-iS18, or any fragment thereof, such that binding occurs between LRP/LR and IgG1-iS18, or any fragment thereof, causing a reduction in Aβ shedding.

It is to be understood that the binding between the mRNA of 37 kDa/67 kDa laminin receptor precursor/high affinity laminin receptor (LRP/LR) (or fragment thereof) and the nucleotide sequence downregulates 37 kDa/67 kDa laminin receptor precursor/high affinity laminin receptor (LRP/LR) such that there are fewer LRP/LR proteins present on the target cell when compared to regular physiological functioning, and wherein said reduction in binding sites leads to reduced concentration of Aβ protein.

Preferably, when binding between the nucleotide sequence, preferably the shRNA of SEQ ID NO: 6 or 7, and the mRNA occurs, such binding is between the nucleotide sequence and LRP mRNA. In a preferred embodiment of the invention the concentration of Aβ protein is reduced. The reduced amount of Aβ causes reduced extracellular Aβ plaque deposition in human and/or animal cells, preferably neuronal cells, therein treating and/or preventing Alzheimer's Disease (AD) via the Aβ neuropathology biochemical pathway. The reduction in Aβ concentration may be a reduction relative to Aβ concentration in a normal healthy human or animal, or it may be a reduction relative to Aβ concentration in a human or animal suffering from AD.

In accordance with a second aspect of this disclosure there is provided a biopharmaceutical agent including (i) a 37 kDa/67 kDa laminin receptor precursor/high affinity laminin receptor (LRP/LR) and/or a fragment thereof and/or (ii) a transfecting agent for expressing a 37 kDa/67 kDa laminin receptor precursor/high affinity laminin receptor (LRP/LR) and/or a fragment thereof, for use in decreasing concentration of tau (τ) protein and/or phosphorylated tau (τ) protein in a target cell of a human or animal subject having Alzheimer's Disease (AD), wherein LRP/LR and/or the fragment thereof being for administration to a target cell of a subject in need thereof. The target cells may typically include brain cells of a human or animal having AD. The target cells may include nerve cells. The target cells may include neurons and glia. The glia may include ependymal cells, astrocytes, microglial cells, and oligodendrocytes.

The biopharmaceutical agent may include a delivery means. The delivery means may be included into, dispersed, or surrounded by a pharmaceutical carrier. The pharmaceutical carrier may be for parenteral or non-parenteral administration to the subject. The delivery means may be adapted to facilitate site specific delivery. The pharmaceutical carrier may be adapted to facilitate site specific delivery. The biopharmaceutical agent included into the delivery means which is in turn included into, dispersed, or surrounded by the pharmaceutical carrier, may together provide for a pharmaceutical composition.

LRP/LR may be encapsulated into the delivery means. Encapsulation may take place using nanoparticles. Typically, the delivery means may be dispersed within or surrounded by the pharmaceutical carrier

The nanoparticles may be functionalized with a first functional group. The first functional group may be at least one of, but not limited to, the following group: chemical, biochemical and biological moieties. The moieties may act as ligands to ensure site specific delivery at the target cell. The pharmaceutical carrier may include a second functional group. The second functional group may be at least one of, but not limited to, the following group: chemical, biochemical and biological moieties. Chemical moieties may include organic, inorganic or a combination of organic and inorganic moieties. Biochemical moieties may include at least one of, but not limited to, the following group: amino acids, peptides, polypeptides, oligopeptides, proteins, enzymes, anti-bodies and RNA or DNA sequences coding for any one of the aforementioned.

Typically, the first functional group in use acts as a ligand to facilitate site specific delivery of the delivery means. It is to be understood that the functional groups may differ depending different cells in the brain. In use, the first functional group bonds with, or joins to, or conjugates with, or associates with, the target site. The second functional group of the pharmaceutical carrier may further aid site specific delivery.

The biopharmaceutical agent or pharmaceutical composition may be provided for parenteral or non-parenteral administration to the subject.

Non-parenteral administration may include at least one of, but not limited to, the following group: oral, nasal, rectal, vaginal, optical and transdermal administration. Typically, non-parenteral administration may be oral. Parenteral administration may include at least one of intravenous, subcutaneous and intramuscular administration. Typically, parenteral administration may be intravenous.

The biopharmaceutical agent when for non-parenteral delivery may be adapted to cross the blood brain barrier.

The biopharmaceutical agent may further include an anti-oxidant such that in use at the target cell the anti-oxidant scavenges reactive oxygen species.

The biopharmaceutical agent may further include an active pharmaceutical ingredient (API). The API may include at least one of, but not limited to, the following group of anti-Alzheimer's Disease drugs: donepezil, rivastigmine, galantamine, memantine and combinations thereof. The Applicant envisages other anti-AD drugs.

LRP/LR may comprise a peptide/protein sequence listing as set forth in SEQ ID NO: 1 or SEQ ID NO: 2, or a fragment thereof as set out herein above, and/or a fragment thereof as set forth in SEQ ID NO: 4 or SEQ ID NO: 5. FLAG may comprise a peptide/protein sequence listing as set forth in SEQ ID NO:3, as defined herein above.

The pharmaceutical carrier may be a polymeric carrier matrix. The polymeric carrier matrix may include polymeric nanoparticles. The polymeric nanoparticles may include synthetic or natural polymers. The polymeric nanoparticles may be biodegradable to provide in use a reduced risk of an immunogenic response to said polymeric nanoparticles. The polymeric nanoparticles may be biocompatible to mitigate risk of any immunogenic response to said polymeric nanoparticles.

The polymeric nanoparticles may be stimuli responsive such that in use the nanoparticles undergo a conformational change upon exposure to certain stimuli to facilitate providing the biopharmaceutical agent to its target site. The nanoparticles may be responsive to stimuli including for example: pH, temperature, and electric current.

The polymeric nanoparticles may be hydrophobic or hydrophilic depending on the specific target site.

The polymeric nanoparticles may be crosslinked. Typically, a crosslinking agent is used for crosslinking. However, crosslinking may take place by way of ultra-violet (U.V.) irradiation.

The polymeric nanoparticles may be lyophilized. Lyophilization typically provides porosity to facilitate diffusion of the biopharmaceutical active (in this case LRP/LR or a transfecting agent for LRP/LR) away from the nanoparticle capsule and to the target site.

The polymeric nanoparticles may be any one or more selected from, but not limited to, the following group: eudragit, gum arabic, carrageenan, cellulose, hydroxypropyl cellulose (HPC), methylcellulose (MC), hydroxypropylmethylcellulose (HPMC), polylactic-co-glycolic acid (PLGA), chitin, pectin, amylopectic, natural rubber, polyethylene, polypropylene, polystyrene, polyamide, polyacrylonitrile, polyvinyl chloride, polyvinyl alcohol (PVA), polyethylene glycol (PEG), polyethylene oxide (PEO), poly(D-lactide) (PDLA), polylactic acid (PLLA), polygalacturonate, methylcellulose (polyacetals), poly(ε-caprolactone), phospholipids, polysaccharides, polyanionic polysaccharides, carboxymethyl cellulose, carboxymethyl amylose, chondroitin-6-sulfate, dermatin sulfate, heparin, heparin sulfate, poly(hydroxyethyl methylacrylate), collagen, fibrinogen, albumin. fibrin, acrylamide, hydroxypropyl methacrylamide-based copolymers, polyacrylamide, poly(N-isopropyl acrylamide) (pNIPAAm), polyvinylpyrrolidone, poly(methacrylic acid-g-ethylene glycol), poly(N-2-hydroxypropyl methacrylamide), poly(glycolic acid)(PGA), poly(lactic acid)(PLA), chitosan, poly(2-hydroxyethylmtehacrylate) (HEMA), polyphazene, phosphorylcholine, hyaluronic acid (HA), hydroxyethyl methacrylate (HEMA), methylene-bis-acrylamide (MBAAm), poly(acrylic acid) (PAAc), poly-acrylamide (PAAm), polyacrylonitrile (PAN), polybutylene oxide (PBO), polycaprolactone (PCL), poly(ethylene imine) (PEI), poly(ethyl methacrylate) (PEMA), propylene fumarate (PF), poly(glucosylethyl methacrylate) (PGEMA), poly(hydroxy butyrate) (PHB), poly(hydroxyethyl methacrylate) (PHEMA), poly(hydroxypropyl methacrylamide) (PHPMA), poly(methyl methacrylate) (PMMA), poly(N-vinyl pyrrolidone) (PNVP), poly(propylene oxide) (PPO), poly(vinyl acetate) (PVAc), poly(vinyl amine), chondroitin sulfate, dextran sulfate, polylysine, gelatin, carboxymethyl chitin, dextran, agarose, pullulan, polyesters, PEG-PLA-PEG, PEG-PLGA-PEG, PEG-PCL-PEG, PLA-PEG-PLA, poly(PF-co-EG), poly(PEG/PBO-terephthalate), PEG-bis-(PLA-acrylate), PEG6CDs, PEG-g-poly(AAm-co-vinlyamine), poly(NIPAAm-co-AAc), poly(NIPAAm-co-EMA), PNVP, poly(MMA-co-HEMA), poly(AN-co-allyl sulfonate), poly(biscarboxy-phenoxy-phosphazene), poly(GEMA-sulfate), poly(PEG-co-peptides), alginate-g-(PEO-PPO-PEO), poly(PLGA-co-serine), collagen-acrylate, alginate, alginate-acrylate, poly(HPMA-g-peptide), HA-g-NPAAm, and poly(vinyl methyl ether) (PVME), and/or derivatives of any one or more of the aforementioned.

In an example embodiment of the disclosure the polymeric nanoparticles may be poly(lactic-co-glycolic acid) (PLGA) nanoparticles.

PLGA is biodegradable, biocompatible and can cross the blood brain barrier (BBB).

The biopharmaceutical agent may further comprise an inhibitor of cytochrome P450 3A4 (CYP3A4). The inhibitor of cytochrome P450 3A4 (CYP3A4) may be selected from the group consisting of polyethylene glycol, polyamine, polymethyl methacrylate and derivatives thereof, wherein the inhibitor is present in an amount which is effective to substantially inhibit the biopharmaceutical and/or the API from being pre-systemically metabolized resulting in greater bioavailability of the biopharmaceutical and/or API.

The biopharmaceutical agent may further comprise a P-glycoprotein (P-gp) efflux pump inhibitor.

In a certain embodiment, wherein the biopharmaceutical agent is formulated for oral delivery it may further include a coating there around, preferably an enteric coating. The coating, in use, prevents degradation of the biopharmaceutical agent in the stomach.

The coating may include the cytochrome P450 3A4 (CYP3A4) inhibitor and/or the P-glycoprotein (P-gp) efflux pump inhibitor.

LRP/LR may comprise a peptide/protein sequence listing as set forth in SEQ ID NO: 1 or SEQ ID NO: 2, or a fragment thereof. LRP/LR may comprise a peptide/protein sequence listing having at least 80% homology to the sequences as set forth in SEQ ID NO: 1 or SEQ ID NO: 2, or a fragment thereof. LRP/LR may comprise homologs or fragments thereof, and homologs of the fragments, wherein LRP LR may comprise a peptide/protein sequence listing as set forth in SEQ ID NO: 1 or SEQ ID NO: 2.

SEQ ID NO: 1 may be a peptide/protein sequence for human LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTATQPEV
ADWSEGVQVPSVPIQQFPTEDWSAQPATEDWSAAPTAQATEWVGATTDW
S

SEQ ID NO: 2 may be a peptide/protein sequence for mouse (Mus musculus) LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTAAQPEV
ADWSEGVQVPSVPIQQFPTEDWSAQPATEDWSAAPTAQATEWVGATTEW
S

It is to be understood that LRP/LR is highly conserved and homologs or fragments of SEQ ID NO: 1 and SEQ ID NO: 2, and/or homologs of the fragments may also utilized in order to exercise the disclosure described, illustrated and/or exemplified herein.

The peptide/protein sequence of LRP/LR or a homolog or fragment thereof, or a homolog of the fragment, may be bound to, or bonded with, or joined to, or conjugated with, or associated with, an additional protein sequence, amino acid sequence, peptide, protein, or antibody.

Alternatively and/or additionally, the peptide/protein sequence of LRP/LR may form part of a larger and/or longer peptide/protein sequence. In a certain embodiment of the invention LRP/LR may be may be bound to, or bonded with, or joined to, or conjugated with, or associated with, FLAG protein, such that in use, the LRP/LR may be tagged with FLAG. FLAG protein may include a peptide/protein sequence that includes at least a sequence motif DYKDDDDK (SEQ ID NO:3). The Applicant envisages employing other tags.

An example embodiment of a fragment of the peptide/protein sequence listing is exemplified as SEQ ID NO: 4 corresponding to a fragment of SEQ ID NO:1 from 102 to 295 and/or SEQ ID NO:5 corresponding to a fragment of SEQ ID NO: 2 from 102 to 295.

SEQ ID NO: 4 may be a peptide/protein sequence for a fragment of human LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTATQPEVADWSEGVQVPSVPIQQFPTEDW
SAQPATEDWSAAPTAQATEWVGATTDWS

SEQ ID NO: 5 may be a peptide/protein sequence for a fragment of mouse LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTAAQPEVADWSEGVQVPSVPIQQFPTEDW
SAQPATEDWSAAPTAQATEWVGATTEWS.

The biopharmaceutical agent may further include a means to downregulate LRP/LR causing a downregulation of AP protein to in use provide for a treatment regime that allows for upregulation of LRP/LR and downregulation of LRP/LR either sequentially or concomitantly, as described in the first aspect above. This means to downregulate may include the anti-laminin receptor specific antibody (preferably IgG1-iS18) and/or the nucleotide (preferably shRNA having SEQ ID NO: 6 and/or SEQ ID NO: 7) as provided in the first aspect of this disclosure herein above.

The transfecting agent may be pCIneo-moLRP::FLAG plasmid.

The biopharmaceutical agent may further include a means to downregulate LRP/LR causing a downregulation of Aβ protein to in use provide for a treatment regime that allows for upregulation of LRP/LR and downregulation of LRP/LR either sequentially or concomitantly, as described in the first aspect above. This means to downregulate may include the anti-laminin receptor specific antibody (preferably IgG1-iS18) and/or the nucleotide (preferably shRNA having SEQ ID NO: 6 and/or SEQ ID NO: 7) as provided in the first aspect of this disclosure herein above.

In accordance with a third aspect of this disclosure there is provided a method of treating and/or preventing Alzheimer's Disease (AD), the method including the following steps:
(i). transfecting the cell to produce 37 kDa/67 kDa laminin receptor precursor/high affinity laminin receptor (LRP/LR) and/or a fragment thereof; or
(ii). providing the cell with LRP/LR and/or fragments thereof,
such that in use, Steps (i) or (ii) provide an overexpression of LRP/LR in the target cell together with a concomitant increase in reverse transcriptase of telomerase (hTERT), which in turn results in decreasing concentration of tau (τ) protein and/or phosphorylated tau (τ) protein in a target of a human or animal having Alzheimer's Disease.

The target cells may typically include brain cells of a human or animal having AD. The target cells may include nerve cells. The target cells may include neurons and glia. The glia may include ependymal cells, astrocytes, microglial cells, and oligodendrocytes.

The transfecting agent and/or the LRP/LR and/or the fragment of LRP/LR may be provided as a delivery means, said delivery means may be included into, dispersed, or surrounded by a pharmaceutical carrier, which may together provide a pharmaceutical composition. The pharmaceutical composition being for parenteral or non-parenteral administration to the subject. The delivery means may be adapted to facilitate site specific delivery. The pharmaceutical carrier may be adapted to facilitate site specific delivery.

Non-parenteral administration may include at least one of, but not limited to, the following group: oral, nasal, rectal, vaginal, optical and transdermal administration. Typically, non-parenteral administration may be oral. Parenteral administration may include at least one of intravenous, subcutaneous and intramuscular administration. Typically, parenteral administration may be intravenous.

The transfecting agent may be pCIneo-moLRP::FLAG plasmid.

LRP/LR may comprise a peptide/protein sequence listing as set forth in SEQ ID NO: 1 or SEQ ID NO: 2, or a fragment thereof.

SEQ ID NO: 1 may be a peptide/protein sequence for human LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTATQPEV
ADWSEGVQVPSVPIQQFPTEDWSAQPATEDWSAAPTAQATEWVGATTDW
S

SEQ ID NO: 2 may be a peptide/protein sequence for mouse (Mus musculus) LRP/LR and may have the following sequence:

MSGALDVLQMKEEDVLKFLAAGTHLGGTNLDFQMEQYIYKRKSDGIYII
NLKRTWEKLLLAARAIVAIENPADVSVISSRNTGQRAVLKFAAATGATP
IAGRFTPGTFTNQIQAAFREPRLLVVTDPRADHQPLTEASYVNLPTIAL
CNTDSPLRYVDIAIPCNNKGAHSVGLMWWMLAREVLRMRGTISREHPWE
VMPDLYFYRDPEEIEKEEQAAAEKAVTKEEFQGEWTAPAPEFTAAQPEV
ADWSEGVQVPSVPIQQFPTEDWSAQPATEDWSAAPTAQATEWVGATTEW
S

It is to be understood that LRP/LR is highly conserved and homologs or fragments of SEQ ID NO: 1 and SEQ ID NO: 2, and/or homologs of the fragments may also utilized in order to exercise the invention described, illustrated and/or exemplified herein.

LRP/LR may comprise a peptide/protein sequence listing having at least 80% homology to the sequences as set forth in SEQ ID NO: 1 or SEQ ID NO: 2, or a fragment thereof.

LRP/LR may comprise homologs or fragments thereof, and homologs of the fragments, wherein LRP/LR may comprise a peptide/protein sequence listing as set forth in SEQ ID NO: 1 or SEQ ID NO: 2.

The peptide/protein sequence of LRP/LR or a homolog or fragment thereof, or a homolog of the fragment, may be bound to, or bonded with, or joined to, or conjugated with, or associated with, an additional protein sequence, amino acid sequence, peptide, protein, or antibody. Alternatively, and/or additionally, the peptide/protein sequence of LRP/LR may form part of a larger and/or longer peptide/protein sequence. In a certain embodiment of the invention LRP/LR may be bound to, or bonded with, or joined to, or conjugated with, or associated with, FLAG protein, such that in use, the LRP/LR may be tagged with FLAG. FLAG protein may include a peptide/protein sequence that includes at least a sequence motif DYKDDDDK (SEQ ID NO:3).

It is to be understood that the step of transfecting the cell to produce 37 kDa/67 kDa laminin receptor precursor/high affinity laminin receptor (LRP/LR) and/or a fragment may take place via known procedures in the art, including introduction into the cell of the transfecting agent. The step of transfecting the cell may upregulate LRP/LR to cause overexpression of LRP/LR.

An example embodiment of a fragment of the peptide/protein sequence listing is exemplified as SEQ ID NO: 4 corresponding to a fragment of SEQ ID NO:1 from 102 to 295 and/or SEQ ID NO:5 corresponding to a fragment of SEQ ID NO: 2 from 102 to 295.

SEQ ID NO: 4 may be a peptide/protein sequence for a fragment of human LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTATQPEVADWSEGVQVPSVPIQQFPTEDW
SAQPATEDWSAAPTAQATEWVGATTDWS

SEQ ID NO: 5 may be a peptide/protein sequence for a fragment of mouse LRP/LR and may have the following sequence:

RFTPGTFTNQIQAAFREPR
LLVVTDPRADHQPLTEASYVNLPTIALCNTDSPLRYVDIAIPCNNKGAH
SVGLMWWMLAREVLRMRGTISREHPWEVMPDLYFYRDPEEIEKEEQAAA
EKAVTKEEFQGEWTAPAPEFTAAQPEVADWSEGVQVPSVPIQQFPTEDW
SAQPATEDWSAAPTAQATEWVGATTEWS.

The method extends generally to upregulation of LRP/LR expression in the target cells.

The method of the third aspect of this disclosure may include a Step (iii), wherein Step (iii) includes:
(iii) continuing and/or repeating Steps (i) and/or Step (ii) for a first period of time in human or animal subject having Alzheimer's Disease (AD).

The Step (iii) may take place weekly for the first period of time. The Step (ii) may take place daily for the first period of time.

The Step (iii) may take place weekly for the first period of time. The Step (iii) may take place daily for the first period of time. The Step (iii) may take place daily, weekly, monthly or any period of time in between and including the aforementioned. The first period of time may be at least six (6) months. The first period of time may be at least 12 months.

The method of the first aspect of this disclosure may include a Step (iv), wherein Step (iv) includes:
(iv) down regulation of LPR/LR prior to any one of Steps (i), (ii) and (iii) being completed, alternatively after any of Steps (i), (ii) and (iii) being completed, further alternatively together with any one of Steps (i). (ii), and (iii).

The Step (iv) provides for combination treatment of Alzheimer's Disease by providing the human or animal subject anti-37 kDa/67 kDa laminin receptor (LRP/LR) specific antibody (or fragment thereof) such that binding occurs between a surface epitope of 37 kDa/67 Kda laminin receptor (LRP/LR) and the anti-37 kDa/67 kDa laminin receptor (LRP/LR) specific antibody (or fragment thereof) causing a decrease in the concentration of amyloid beta (Aβ) peptide in the human or animal.

Step (iv) may alternatively, or additionally, include providing to the human or animal subject a nucleotide sequence, preferably shRNA of SEQ ID NO: 6 and/or SEQ ID NO: 7, such that binding occurs between mRNA of 37 kDa/67 kDa laminin receptor (LRP/IR) and the shRNA causing downregulation of the 37 kDa/67 kDa laminin receptor (LRP/LR) which in turn causes a decrease in the concentration of amyloid beta (Aβ) peptide in the human or animal.

SEQ ID NO: 6 may be gcucgugcaa uuguugccau u. The sequence may be for Homo sapiens RNA.

SEQ ID NO: 7 may be ggcagugacc aaggaggaau u. The sequence may be for Homo sapiens RNA.

Step (iv) downregulates LRP/LR which causes a decrease in Aβ. The reduction in Aβ shedding may be a reduction relative to Aβ shedding in a normal healthy human or animal, or it may be a reduction relative to Aβ shedding in a human or animal suffering from AD.

Step (iv) is counterintuitive to provide before, during or after any one or all of Steps (i), (ii) and (iii). Steps (i) to (iii) upregulate LRP/LR causing a decrease in concentration of tau (τ) protein and/or phosphorylated tau (τ) protein. It is known that the biochemical pathways associated with Aβ and tau (τ) neuropathologies are substantially dissimilar and treatment methodologies successfully targeting the one will not necessarily be successful on the other.

Concomitant and/or sequential treatment of the Aβ and tau (τ) neuropathologies via the upregulation and downregulation of LRP/LR as described herein provides a surprising and unexpected solution to ameliorating disadvantages of both the Aβ and tau (τ) neuropathologies. The skilled person would not have considered upregulation of LRP/LR to ameliorate Alzheimer's Disease (AD), and would certainly not have considered a combination therapy including the concomitant and/or sequential upregulation and downregulation of LRP/LR.

It is to be understood in regard to the downregulation using an anti-37 kDa/67 kDa laminin receptor (LRP/LR) specific antibody, that the binding between the surface epitope of 37 kDa/67 kDa laminin receptor precursor/high affinity laminin receptor (LRP/LR) (or a fragment thereof) and antibody causes a reduction in Aβ shedding.

The anti-37 kDa/67 kDa laminin receptor (LRP/LR) specific antibody, or fragment thereof, may be raised against the cell surface protein being 37 kDa/67 kDa laminin receptor precursor/high affinity laminin receptor (LRP/LR). In a preferred embodiment the antibody is raised against LRP/LR or against a protein having 80% or greater homology with LRP/LR. The antibody, or fragment thereof, may be a F(ab′)2 fragment, a Fab fragment scFv, a bi-specific scFv, a tri-specific scFv, a single chain or tandem diabody, a single domain antibody (dAb), a minibody or a molecular recognition unit (MRU). Furthermore, the antibody, or fragment thereof, may be monovalent, bivalent or multivalent. The antibody, or fragment thereof, may additionally comprise at least one further antigen-interaction site and/or at least one further effector domain. In a preferred embodiment of the invention, the antibody or fragment thereof may be an anti-laminin receptor specific antibody, preferably an anti-LRP/LR specific antibody, further preferably IgG1-iS18.

In a preferred embodiment of the disclosure, the method for reducing Aβ shedding caused by the proteolytic cleavage of APP by beta (β) and gamma (γ) secretases, the method comprises contacting LRP/LR with IgG1-iS18, or any fragment thereof, such that binding occurs between LRP/LR and IgG1-iS18, or any fragment thereof, causing a reduction in Aβ shedding. It is to be understood that the binding between the mRNA of 37 kDa/67 kDa laminin receptor precursor/high affinity laminin receptor (LRP/LR) (or fragment thereof) and the nucleotide sequence downregulates 37 kDa/67 kDa laminin receptor precursor/high affinity laminin receptor (LRP/LR) such that there are fewer LRP/LR proteins present on the target cell when compared to regular physiological functioning, and wherein said reduction in binding sites leads to reduced concentration of A protein.

Preferably, when binding between the nucleotide sequence, preferably the shRNA of SEQ ID NO: 6 or 7, and the mRNA occurs, such binding is between the nucleotide sequence and LRP mRNA. In a preferred embodiment of the invention the concentration of Aβ protein is reduced. The reduced amount of Aβ causes reduced extracellular Aβ plaque deposition in human and/or animal cells, preferably neuronal cells, therein treating and/or preventing Alzheimer's Disease (AD) via the Aβ neuropathology biochemical pathway. The reduction in Aβ concentration may be a reduction relative to Aβ concentration in a normal healthy human or animal, or it may be a reduction relative to Aβ concentration in a human or animal suffering from AD.

In accordance with a fourth aspect of this disclosure there is provided use of (i). 37 kDa/67 kDa laminin receptor precursor/high affinity laminin receptor (LRP/LR) and/or a fragment thereof, or (ii). a transfecting agent for the expression of LRP/LR, in the manufacture of a pharmaceutical composition for use in decreasing concentration of tau (τ) protein and/or phosphorylated tau (τ) protein in a target cell of a human or animal subject having Alzheimer's Disease (AD). The (i). 37 kDa/67 kDa laminin receptor precursor/high affinity laminin receptor (LRP/LR) and/or a fragment thereof, or (ii). a transfecting agent for the expression of LRP/LR, are as described herein above. The pharmaceutical composition may be as described herein above.

There is provided any one or more of the first to fourth aspects of this disclosure substantially as herein described as described and/or exemplified and/or illustrated herein.

### BRIEF DESCRIPTION

Embodiments of the disclosure will be described below by way of example only and with reference to the accompanying drawings in which:

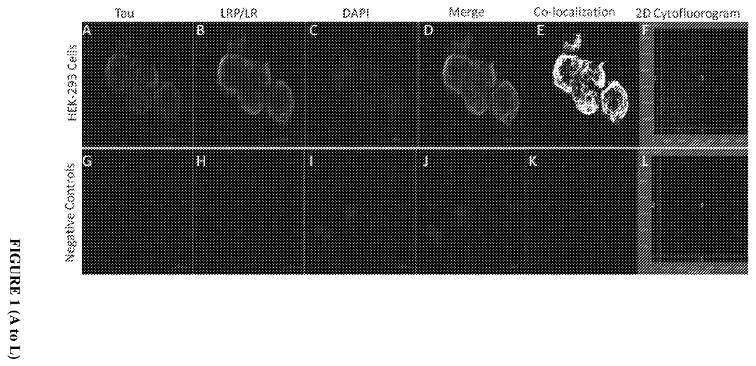

FIG. 1 shows LRP/LR and tau (τ) localization and co-localization within HEK-293 cells. This figure illustrates the co-localization between LRP/LR (green frame B) and tau (τ) (red frame A). A) Endogenous tau levels in HEK-293 cells. Tau localizes to the cytosol. B) Endogenous LRP/LR levels in HEK-293 cells. LRP/LR localizes to the cytosol, nucleus and cell surface. C, I) Nuclei are stained with DAPI (blue). Co-localization occurs between LRP/LR and tau in the perinuclear compartment, represented by yellow fluorescence in the merged image (D), as white areas (E) and as fluorescence in the third quadrant of the 2D cytofluorogram (F). G) CF 647™ only control. H) FITC only control. The merged image (J), co-localization image (K) and 2D cytofluorogram (L) show that there is no co-localization between the negative controls. All images were taken with the Zeiss LSM 780 microscope with the addition of Airyscan™ at a 630× magnification. Scale bars represent 20 sim.

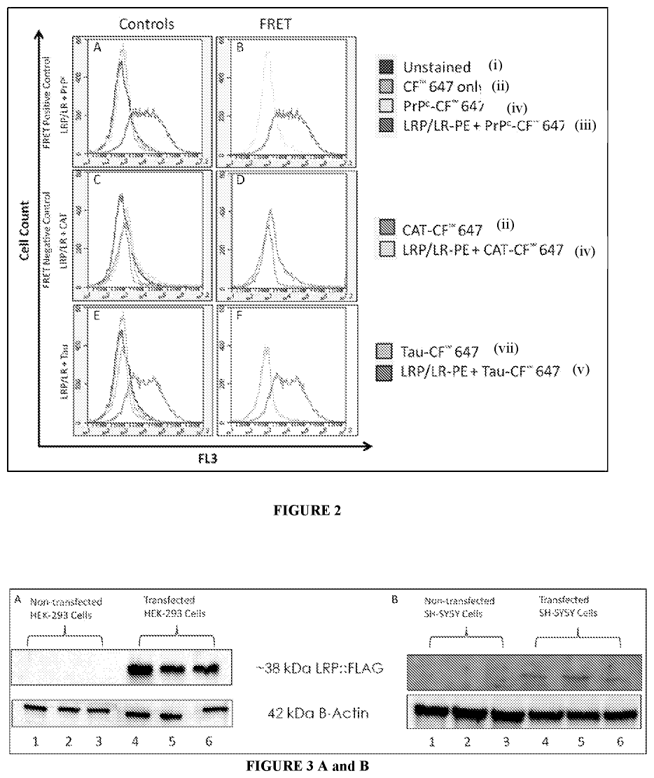

FIG. 2 shows flow cytometric analysis of FRET between intracellular LRP/LR and tau (τ). The fluorescence intensity of the unstained HEK-293 cells (dark blue histogram—i) was superimposed with that of cells labelled with the CF™ 647 secondary antibody only (yellow histogram—ii) as well as with cells in which the proteins of interest; PrP^{c}. CAT and tau, were labelled with CF™ 647 (A, C, E). B) Co-labelling of LRP/LR-PE and PrP^{c}-CF™ 647 (positive control) (pink histogram—iii). D) Co-labelling of LRP/LR-PE and CAT-CF™ 647 (negative control) (green histogram—iv). F) Co-labelling of LRP/LR-PE and Tau-CF™ 647 (red histogram—v). Each panel is a representative image. Three biological replicates were conducted.

FIG. 3 shows overexpression of LRP::FLAG in HEK-293 and SH-SY5Y cells transfected with the pCIneo-moLRP-FLAG plasmid. Lanes 1-3: non-transfected cells. Lanes 4-6: transfected cells. A) LRP::FLAG (HRP) was detected in transfected HEK-293 cells but not non-transfected HEK-293 cells B) LRP::FLAG (Cy3) was detected in transfected SHSY-5Y cells but not non-transfected SH-SY5Y cells. B-Actin (HRP) was used as a loading control, n=3 biological repeats.

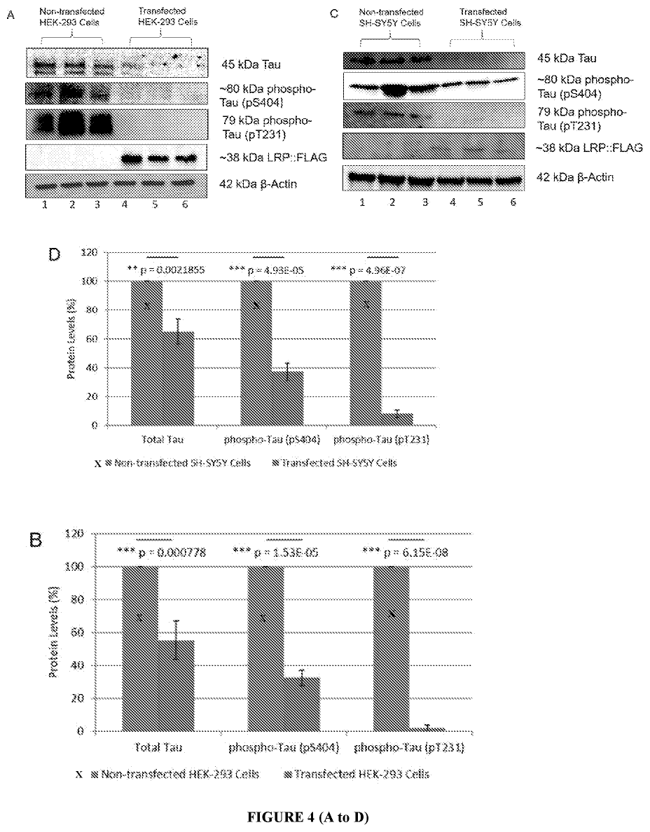

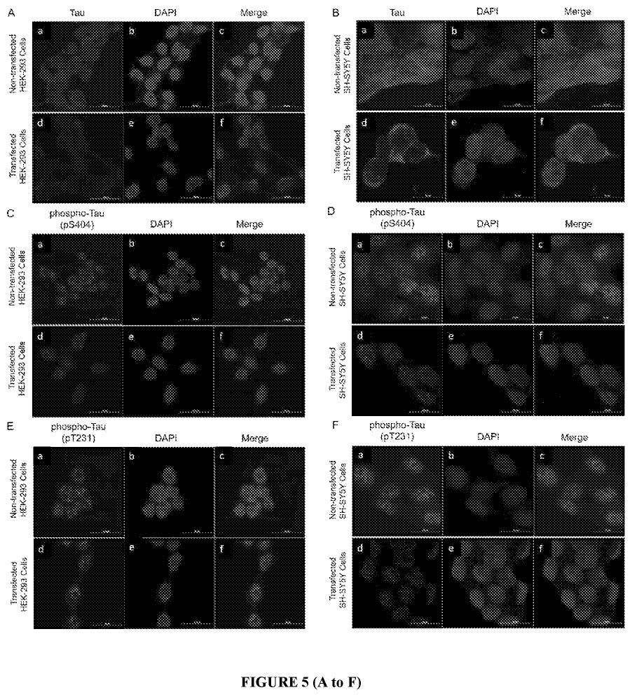

FIG. 4 shows overexpression of LRP::FLAG in HEK-293 and SH-SY5Y cells decreases levels of total Tau as well as phospho-Tau. A. B) A 45% decrease in total Tau, 68% decrease in phospho-Tau (pS404) and 98% decrease in phospho-Tau (pT231) was observed in transfected HEK-293 cells. C, D) A 35% decrease in total Tau, 63% decrease in phospho-Tau (pS404) and 92% decrease in phospho-Tau (pT231) was observed in transfected SH-SY5Y cells. A. C) HEK-293 and SH-SY5Y cells were confirmed to be overexpressing LRP::FLAG after stable transfection with the pCIneo-moLRP-FLAG plasmid. LRP::FLAG was detected in transfected HEK-293 cells only (A). LRP::FLAG was detected in transfected SHSY-5Y cells only (C). A, C) β-Actin was used as a loading control. Densitometric analysis performed was relative to the non-transfected HEK-293 (B) and SH-SY5Y (C) cells which were set to 100%. Error bars represent standard deviation, n=3 biological repeats. *p: 0.05. **p: 0.01, ***p: 0.001; Student's i-test.

FIG. 5 shows intracellular expression of tau and phospho-Tau in transfected and non-transfected HEK-293 and SH-SY5Y cells, b, e) Nuclei are stained with DAPI (blue). A. B) Expression of total tau decreases in transfected HEK-293 and transfected SH-SY5Y cells. Endogenous levels of total tau (red) (a). Levels of total tau (red) after transfection with pCIneo-moLRP-FLAG plasmid (d). Merge of total tau with DAPI staining (c, f). C. D) Expression of phospho-Tau (pS404) is decreased in transfected HEK-293 and transfected SH-SY5Y cells. Endogenous levels of phospho-Tau (pS404) (red) (a). Levels of phospho-Tau (pS404) (red) after transfection with pCIneo-moLRP-FLAG plasmid (d). Merge of phospho-Tau (pS404) with DAPI staining (c, f). E, F) Expression of phospho-Tau (pT231) is decreased in transfected HEK-293 and transfected SH-SY5Y cells. Endogenous levels of phospho-Tau (pT231) (red) (a). Levels of phospho-Tau (pT231) (red) after transfection with pCIneo-moLRP-FLAG plasmid (d). Merge of phospho-Tau (pT231) with DAPI staining (c, f). All images were taken at 630× magnification with the addition of Airyscan™. Scale bars represent 20 μm.

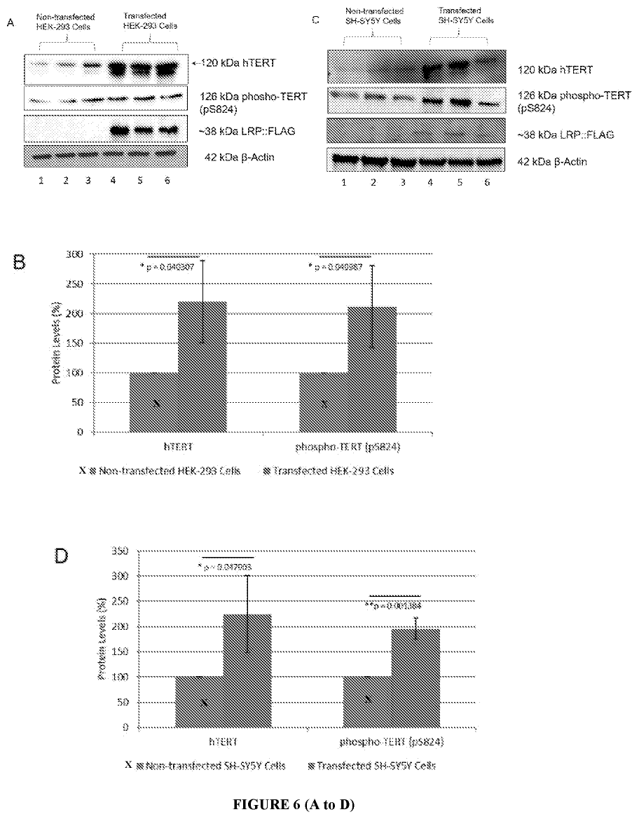

FIG. 6 shows LRP::FLAG overexpression increases hTERT and phospho-TERT levels in HEK-293 and SH-SY5Y cells. A, B) A 120% increase in hTERT and 112% increase in phospho-TERT was observed in transfected HEK-293 cells. C, D) A 125% increase in hTERT and 96% increase in phospho-TERT was observed in transfected SH-SY5Y cells. β-Actin was used as a loading control. Densitometric analysis performed was relative to the non-transfected HEK-293 (B) and SH-SY5Y (D) cells which were set to 100%. Error bars represent standard deviation, n=3 biological repeats. *p: 0.05. **p: 0.01, ***p: 0.001; Student's i-test.

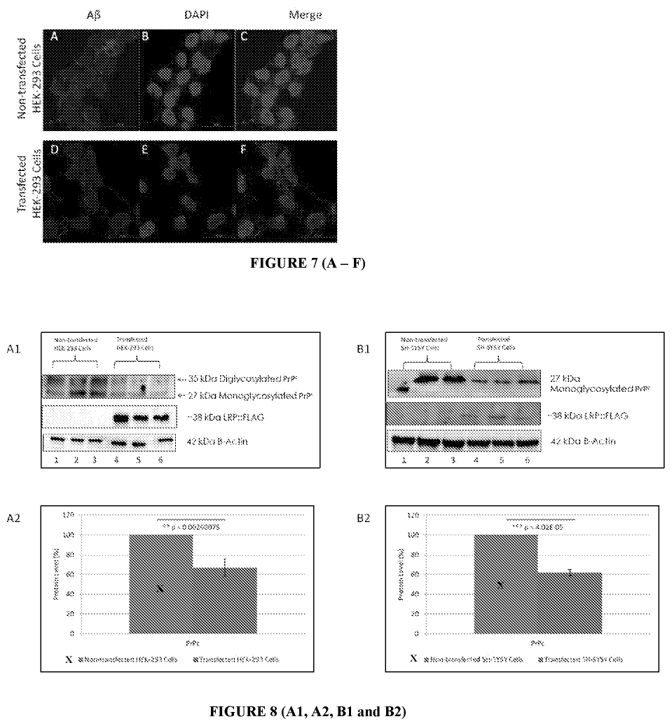

FIG. 7 shows intracellular expression of Aβ₄₂ in transfected and non-transfected HEK-293 cells. Expression of Aβ₄₂ decreases in transfected HEK-293 cells. A) Endogenous levels of Aβ₄₂. Aβ₄₂ localizes to the cytosol and cell surface. D) Levels of Aβ₄₂ after transfection with pCIneo-moLRP-FLAG plasmid. B, E) Nuclei are stained with DAPL C, F) Merge of Aβ₄₂ with DAPI staining. All images were taken with the Zeiss LSM 780 microscope with the addition of Airyscan™ at a 630× magnification. Scale bars represent 20 μm.

FIG. 8 shows overexpression of LRP::FLAG in HEK-293 and SH-SY5Y cells decreases PrP^{c} levels. A1, B1) Lanes 1-3: non-transfected cells. Lanes 4-6: transfected cells. B-Actin (HRP) was used as a loading control. Densitometric analysis performed was relative to the non-transfected HEK-293 (A2) and SH-SY5Y (B2) cells which were set to 100%. Error bars represent standard deviation, n=3 biological repeats. *p: 0.05, **p: 0.01, ***p: 0.001; Student's t-test.

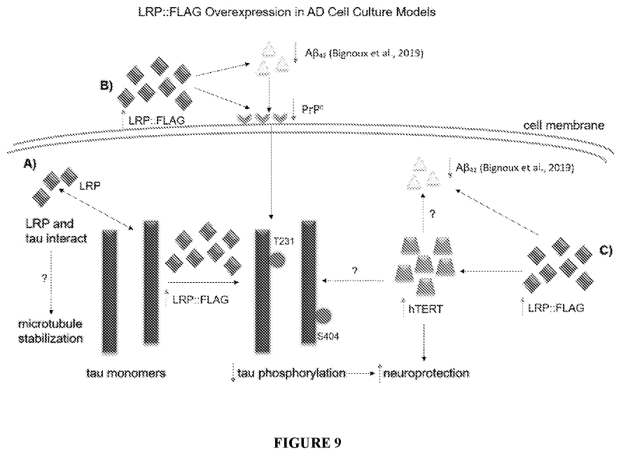

FIG. 9 shows a proposed mechanisms of LRP::FLAG overexpression in AD cell culture models. A) Direct interaction between LRP and tau: an increase in cytoplasmic LRP::FLAG could stabilize microtubules through the direct interaction of LRP and tau. B) Aβ₄₂-PrP^{c}-hyperphosphorylated tau cascade: LRP::FLAG overexpression decreases the levels of Aβ₄₂ and PrP^{c} which may contribute to decreased phosphorylated tau. C) Increased hTERT: LRP::FLAG overexpression increases hTERT levels which conveys neuroprotection as it is able to protect against oxidative stress. Increased hTERT levels may also decrease the level of intercellular Aβ₄₂ and phosphorylated tau, however, the exact mechanism is unknown. LRP::FLAG overexpression also results in decreased senescent marker proteins γH2AX and β-galactosidase. Dotted lines and question marks indicate an unknown mechanism.

### DETAILED DESCRIPTION

The content of the Summary above is fully repeated herein by way of reference and to avoid unnecessary repetition.

It is to be understood that LRP/LR is highly conserved and homologs or fragments of SEQ ID NO: 1 and SEQ ID NO: 2, and/or homologs of the fragments may also utilized in order to exercise the disclosure described, illustrated and/or exemplified herein.

Substantially identical sequences may also be employed. As used herein, a substantially identical sequence is an amino acid or nucleotide sequence that differs from a reference sequence only by one or more conservative substitutions, or by one or more non-conservative substitutions, deletions or insertions located at positions of the sequence that do not destroy or substantially reduce the activity of one or more of the expressed polypeptides or of the polypeptides encoded by the nucleic acid molecules.

In the examples below, the transfecting agent is be pCIneo-moLRP::FLAG plasmid. It is to be understood that another transfecting agent for use in the upregulation of LRP/LR expression in the target cells is envisaged by the Applicant. Detailed example embodiments of the disclosure, which are not limiting to the scope of the disclosure, are provided herein below in the Examples.

The two primary neuropathologies for AD have vastly different biochemical pathways and manifest in very different locations. The aggregation of amyloid beta (Aβ) is extracellular, while neurofibrillary tangles are from intracellular accumulation of hyperphosphorylated tau (τ). Consequently, there is no reasonable expectation of success to the person skilled in the art of that a certain treatment protocol would be successful in targeting tau (τ) when it has been shown to be successful in treating aggregation of amyloid beta (Aβ). Similarly, unsuccessful protocols targeting Aβ are not expected to be successful in regard to tau (τ). The disclosure described herein is surprising and unexpected.

### Tau (τ)

Tau (τ) is a microtubule-associated protein that functions to stimulate the assembly of tubulin into microtubules. Furthermore, tau (τ) binds to and stabilizes the microtubules once they are assembled. Since these microtubules are situated along the cytoskeletal tracks they mediate vital cell processes, such as the transport of nutrients and neurotransmitters. There are six different isoforms of tau (τ) that are present in the adult human brain and they stimulate microtubule assembly with varying efficiency. In healthy cells, the level of tau (τ) phosphorylation is highly modulated and regulates microtubule plasticity, motor activity and axonal transport, and neurite outgrowth.

In AD, however, tau (τ) is hyperphosphorylated and forms tangles, thereby disrupting the internal structure of neuronal cells. In addition, phosphorylation of tau (τ) at specific sites, such as S262, S356 and S422, inhibits the ability of the hyperphosphorylated tau (τ) to be recognized by proteases, thereby sparing it from proteasomal degradation. Therefore, the hyperphosphorylation of tau (τ) is able to induce the AD pathology in multiple ways. Firstly, this hyperphosphorylation can induce the missorting of tau (τ) from the axons to the dendritic spines, resulting in inhibition of transport and loss of spines. Hyperphosphorylated tau (τ) is also known to interact with the kinesin-associated protein, c-Jun N-terminal kinase-interacting protein (JIP1) and impair the formation of the kinesin complex. This complex is important in mediating axonal transport. Lastly, neurofibrillary tangles are formed when this hyperphosphorylation causes tau (τ) to dissociate from microtubules and diffuse into the cytoplasm. This causes the cytoskeletal tracks to disintegrate, thereby inhibiting vital cell processes, such as the transport of nutrients and neurotransmitters, which ultimately leads to cell death. Therefore, the hyperphosphorylation of tau (τ) negatively impacts synaptic health, plasma membrane cell signaling and the protection of DNA from cell stressors, all of which may contribute to the development or progression of neurodegeneration. The degree of dementia observed has been correlated with the number of neurofibrillary tangles present within the brain tissue.

There are approximately 80 putative serine, threonine or tyrosine phosphorylation sites on the tau (τ) protein. As a result, the normal level of tau phosphorylation is the result of a dynamic interplay between tau kinases and tau phosphatases. Tau kinases, such as glycogen synthase kinase 3 (GSK3) and cyclin-dependent protein kinase 5 (CDK5) work to phosphorylate tau (τ) while phosphatases, such as protein phosphatase 2A and 2B (PP2A and PP2B), reverse this phosphorylation. In tau (τ) pathologies, prior phosphorylation of tau (τ) by CDK5 enhances its subsequent phosphorylation by GSK3. GSK3 then phosphorylates residues which affect the binding of tau (τ) to microtubules. The residues threonine-231, serine-396 and serine-404 are particularly important in this role. Furthermore, the phosphorylation of tau (τ) by GSK3 may be required in order for tau polymers to form. In AD, CDK5 may be upregulated, while PP2A is often downregulated. Since PP2A has broad substrate specificity, the downregulation of PP2A can result in the hyperphosphorylation of several other neuronal proteins, such as p-tubulin and β-catenin, which further exacerbates neuronal damage by inhibiting the assembly of these molecules.

### Amyloid Beta (Aβ)

Although Aβ is a normal physiological peptide and present in healthy cells, its functions are not well understood, but it has been implicated in neuronal survival and viability. It is produced by the sequential cleavage of the amyloid precursor protein (APP). In healthy cells, APP can be processed by two distinct pathways, namely non-amyloidogenic and amyloidogenic processing. In the non-amyloidogenic pathway, APP is sequentially cleaved by α-secretase and γ-secretase to yield sAPPα, a soluble non-pathogenic protein, and a peptide called P3. Importantly, the initial cleavage of APP by α-secretase occurs within the Aβ amino acid sequence, thereby preventing the generation of Aβ. However, during amyloidogenic processing, sequential cleavage of APP by β-secretase and γ-secretase yields Aβ and the APP intracellular domain (AICD). In AD, misappropriation of this amyloidogenic pathway or a decline in Aβ clearance causes an increase in the concentration of Aβ peptide resulting in the development of the disease. There is substantial evidence that A is the primary pathological agent of AD; however, there are still limitations to this hypothesis.

The Aβ monomers formed from amyloidogenic processing then misfold, associate and aggregate in various ways. The soluble Aβ₄₂ oligomers, in particular, accumulate in the cell as they have a higher aggregation propensity than the other isoforms. This isoform also has a higher neurotoxicity than the other Aβ isoforms. Thus, plaques form due to this aggregation of Aβ and are responsible for the initial neuronal loss seen in AD. Additionally, these plaques are able to interfere with the inter-neuronal synaptic signaling and induce inflammation of the brain, which activates an immune response. These immune cells will further exacerbate the disease as they breakdown and digest the damaged neurons. In order for AD to progress. Aβ₄₂ needs to interact with lipid membranes and cellular receptors. These Aβ₄₂ oligomers associate with the plasma membrane of neuronal cells resulting in distortion of the membrane and the formation of ion channels. This causes an influx of calcium (Ca²⁺) ions which disrupt synaptic plasticity and enhances mitochondrial permeability, leading to cytotoxicity. Indeed, destabilization of the Ca²⁺ levels triggers free radical formation, lipid peroxidation and apoptosis which could contribute to cytotoxicity.

### Telomerase

Telomerase is a ribonucleoprotein responsible for regulating proliferative potential and preventing senescence in tumorigenic, germline and immortalized cells. It is composed of two essential subunits, among others. In human, these subunits include: hTERC, which contains a telomeric RNA template that the catalytic reverse transcriptase subunit hTERT reads. Together these subunits operate to extend telomeres in a 3′-5′ direction, hTERT is the limiting factor for telomerase activity. This is due to its lower expression levels compared to hTERC, and thus, serves as the major regulator for telomerase activity. This said, telomerase fulfils its core function within the nucleus, where it elongates and maintains telomere length. This maintenance allows not only the continuation of normal cellular processes, but also improves the overall proliferative potential of cells.

Aside from telomere extension, telomerase/hTERT has extra-telomeric functions other than telomere maintenance.

The chromosomal ends that telomerase extends, known as telomeres, are composed of genomic TTAGGG repeats and associated protein structures. These repeats and related proteins together “cap” linear eukaryotic chromosomes for protection. A few of these telomere-related proteins include TRF1, TRF 2 and POT1. These proteins interact with the telomeres and each other to form the “shelterin” complex.

This complex facilitates the folding of telomeres to form a telomere loop (t-loop) to prevent telomere degradation. This said, telomeres and their related proteins fulfil a vital function in protecting against genomic DNA damage. In addition, these telomeric structures also aid to distinguish between normal chromosomes and double stranded breaks. This damage is caused by the imperfect replicating nature of DNA polymerase that generates gaps during lagging strand synthesis.

Additionally. DNA synthesis follows a unidirectional path (3′ to 5′), resulting in DNA ends not being fully replicated, otherwise known as the “end replication problem”. Thus, telomeres as well as their extension prevent the loss of genetic information, ensuring genomic stability and in turn cell viability.

### Non-Limiting Aspects of Disclosure

It has previously been shown in International Pat. Appl. No. PCT/IB20121054968 (WO 2013/042053) that telomerase biology plays a role in Alzheimer's Disease (AD). Here LRP/LR receptor blockage and/or downregulation of LRP/LR using anti-LRP/LR antibodies and shRNAs effectively impeded Aβ shedding. Knock-down of LRP/LR has also been shown to rescue cells from Aβ₄₂-PrP^{c} mediated cytotoxicity.

Interestingly, overexpression of LRP/LR impedes Aβ shedding through interaction with telomerase, and an LRP/LR knockdown in vivo decreased amyloid plaques.

It has been suggested that tau (τ) hyperphosphorylation is dependent on Aβ aggregation, and a decrease in Aβ could aid in the reduction of hyperphosphorylated tau (τ). Consequently, there is a suggestion of an inter-relatedness between amyloid beta and tau despite being biochemically very different.

The Applicant upregulated LRP/LR via transfection of HEK-293 cells using pCIneo-moLRP-FLAG plasmid transfection. The Applicant was surprised when overexpression of LRP::FLAG resulted in a decrease in total tau (τ) and phosphorylated tau (τ) levels in vitro. This decrease in tau (τ) is observed with a concomitant increase in telomerase, and specifically hTERT, activity. This is in contrast to the suggestions and motivations in the prior art.

LRP/LR and tau (τ) were seen to co-localize in the perinuclear compartment and Försters Resonance Energy Transfer (FRET) confirmed that they directly interact with each other. Thereafter. SH-SY5Y and HEK-293 cells were transfected with the pCIneo-moLRP-FLAG plasmid in order to overexpress LRP::FLAG. A decrease in total tau (τ) and phosphorylated tau (τ) was observed in cells overexpressing LRP::FLAG, which was confirmed using western blot analysis and confocal microscopy. This decrease in phosphorylated tau (τ) is indicative of a decrease in hyperphosphorylated tau (τ). There was also a concomitant increase in hTERT levels, determined by western blotting, which could, without being limited to theory, rescue AD-affected cells from cytotoxicity.

Furthermore, the levels of Aβ and PrP^{c}, two tauopathy-related proteins, were also shown to decrease. This is in contrast to the teachings of the prior art wherein LRP/LR knockdown resulted in a decrease in Aβ shedding. This indicates that the mechanisms of action are complicated and not well understood. These proteins are part of a signaling cascade which results in the hyperphosphorylation of tau (τ). Overall, these results suggest that overexpression of LRP::FLAG provides treatment strategy for AD and other tauopathies.

Both in vitro and in vivo studies have shown that the anti-LRP/LR specific antibody, IgG1-iS18, can impede neurodegeneration by reducing Aβ levels in AD. Therefore, it may be beneficial to examine a combinatorial treatment, which otherwise would be counterintuitive, using both LRP::FLAG overexpression and IgG1-iS18 blockade of LRP/LR. This treatment could target more than one aspect of AD, and thereby improve the outcome of the treatment. Delivery mechanisms are also to be explored to ensure hepatic first pass and/or crossing of the blood brain barrier, when desired. Combinatorial treatment is envisaged and includes upregulation and downregulation of LRP/LR either concomitantly or sequentially.

The Applicant believes that the subject matter of the disclosure described herein at least ameliorates one of the disadvantages known in the current state of the art.

### NON-LIMITING EXAMPLES

### Experimental Procedures

### Cell Culture

Cell culture is an in vitro model that attempts to mimic the in vivo conditions in which cells grow, allowing for the investigation into the biochemical and biological processes of the cells. The following cell lines were used:

SH-SH5Y: human neuroblastoma cells from Sigma Aldrich. HEK-293: human embryonic kidney cells from ATCC. SH-SY5Y cells are neuronal cells that are commonly used to investigate neurodegenerative diseases while HEK-293 cells were used as a control since they express neuronal markers and have detectable levels of telomerase activity.

The cells were cultured in Dulbecco's Modified Eagle Medium (DMEM) with high glucose (4.5 g/1) and 4 mM L-Glutamine (Hyclone): Ham's F12 with 1 mM L-Glutamine (Hyclone) in a 1:1 ratio. In addition, the media was supplemented with 15% foetal bovine serum (FBS)(Hyclone) and 2% penicillin/streptomycin (Biowest). The cells were incubated in a humidified incubator at 37° C. with 5% CO₂ to permit optimal proliferation.

In order to maintain the cell line, the cells were passaged when they reached a confluency of 70-80%. First, the cells were washed with phosphate buffered saline (PBS) and then incubated with trypsin at 37° C. for 5 minutes in order to detach the cells from the cell culture flask. Thereafter, fresh media was added for total volume of 10 ml, in order to inactivate the trypsin. Cells were then split in a 1:2 ratio (SH-SH5Y) or 1:10 ratio (HEK-293) and, again, fresh media was added to a total volume of 10 ml.

In addition, cell stocks were prepared in order to cryopreserve cells. After trypsinization, cells were centrifuged at 5000 rpm for 10 minutes. Cell pellets were then resuspended in a mixture of 80% FBS and 20% dimethyl sulfoxide (DMSO) (Merck Millipore) and stored at −70° C. Cell harvesting was performed on cells with 70-80% confluency in order to collect samples for downstream applications. After trypsinization cells were centrifuged at 5000 rpm for 10 minutes. Thereafter, the pellets were stored at −20° C. until required.

Confocal Microscopy with the Addition of Airyscan™

Confocal microscopy is a qualitative technique that allows for the localization of fluorescently labelled proteins to be determined. This technique was performed to determine whether LRP/LR and tau (τ) co-localize and to confirm the co-localization between LRP/LR and hTERT.

Cells were grown to a confluency of 70-80% and 500 μl was seeded into a total volume of 3 ml onto microscope coverslips within a 6-well plate. Thereafter, cells were incubated for 24 hours in a humidified incubator in order to reach 50-70% confluency. All further steps were performed with gentle agitation. Cells were fixed with 4% formaldehyde (VWF) in PBS for 20 minutes. In order to view the intracellular structure of cells, they were permeabilized with 0.25% Triton X-100 (Sigma Aldrich) in PBS for 20 minutes. Cells were then blocked in 0.5% bovine serum albumin (BSA) (Amresco) in PBS for 20 minutes, and thereafter primary antibody (1:200 in 0.5% BSA in PBS) was added. The cells were incubated overnight at 4° C. The following day, the coverslips were washed with PBS for 5 minutes and then incubated with the appropriate fluorescein isothiocyanate (FITC)-coupled or the CF™ 647-coupled secondary antibody (1:500 in 0.5% BSA in PBS) and incubated for an hour in the dark. The primary and secondary antibodies used are listed in Table 1. The cells were then counterstained with 0.1 μg/ml 4′,6-diamidino-2-phenylindole (DAPI) (Sigma Aldrich) for 5 minutes in the dark in order to stain the nuclei. Coverslips were then washed and mounted onto clean microscope slides using Fluoromount™ Aqueous Mounting Medium (Sigma Aldrich). The slides were incubated at room temperature for 1.5 hours to allow the mounting medium to set. All images were acquired at room temperature using the Zeiss LSM 780 confocal microscope at 630× magnification. In addition, Airyscan™ was used to increase the resolution from 200 nm to 140 nm.

TABLE 1
Antibodies used for confocal microscopy

| Target | | |
|---|---|---|
| Protein | Primary Antibody | Secondary Antibody |
| LRP/LR | Human anti-LRP/LR | Anti-human IgG-FITC |
| | (IgG1-iS18) | (Sigma Aldrich: F9512) |
| Total Tau | Mouse anti-Tau | Anti-mouse IgG-CF ™ 647 |
| (Tau-5) | (Tau-5) | (Sigma Aldrich: SAB4600182) |
| | (EMD): MAB361) | |
| Phospho- | Rabbit anti-p-Tau | Anti-rabbit IgG-CF ™ 647 |
| Tau (pS404) | (S404) (Cell | (Sigma Aldrich: SAB4600184) |
| | Signalling: 20194S) | |
| PHF-Tau | Mouse anti-Phospho-Tau | Anti-mouse IgG-CF ™ 647 |
| (pT231) | (Thr231) (AT180) | (Sigma Aldrich: SAB4600182) |
| | (Invitrogen: MN1040) | |
| hTERT | Mouse anti-hTERT | Anti-mouse IgG-CF ™ 647 |
| | (Santa Cruz | (Sigma Aldrich: SAB4600182) |
| | Biotechnology: | |
| | sc-77511) | |
| Aβ₄₂ | Rabbit, anti-beta- | Anti-rabbit IgG- FITC |
| | Amyloid (1-42) | (Sigma Aldrich: F0382 |
| | (Abcam: ab39377) | |

### Flow Cytometric Analysis of Försters Resonance Energy Transfer (FRET)

FRET is a very sensitive technique that is used to assess protein interactions in vitro. In this technique, a non-radiative transfer of energy will only occur if the fluorochromes are within 1-10 nm of each other. This distance-dependent transfer of energy occurs between a donor and acceptor molecule. In this experiment the donor/acceptor pair phycoerythrin (PE)/allophycocyanin (APC) was used to immunolabel the proteins of interest. PE is excited by the 488 nm argon laser and emits at 575 nm. Thus, it may be detected using the FL2 filter set of the Accuri C6 Flow Cytometer (BD Biosciences). CF™ 647, which is an APC contemporary, is excited by the 650 nm neon/helium laser and emits at 660 nm, and can be detected using the FL4 filter set. The presence of FRET between the proteins of interest was evaluated using the FL3 filter set. Within this channel, excitation is achieved with the 488 m argon laser and emission is detected at 660 mu. CF™ 647 is not excited by the 488 nm laser and therefore does not exhibit fluorescence within this channel. However, if CF™ 647 is in close proximity to PE, it may be indirectly excited via FRET resulting in enhanced fluorescence emission in FL3. Thus, FL3 is considered the optimal channel for PE/APC FRET detection.

HEK-293 cells were incubated in serum-free media for 3 hours prior to assessment. Cells were then detached and harvested by centrifugation at 1200 rpm for 10 minutes. The cells were washed twice with PBS. All wash steps were carried out at 5000 rpm for 5 minutes. Cells were then incubated in 4% formaldehyde at 4° C. for 20 minutes in order to fix them. Thereafter, cells were centrifuged at 5000 rpm for 10 minutes to remove the formaldehyde. Since tau is an intracellular protein, the cells were permeabilized by incubating them in 0.1% Triton X-100 in PBS at 4° C. for 20 minutes. Again, the cells were centrifuged at 5000 rpm for 10 minutes in order to remove the Triton X-100. Cells were washed once with PBS and then blocked in 0.5% BSA in PBS for 10 minutes at room temperature. Thereafter, primary antibodies (1:100 in 0.05% BSA in PBS) were added and the cells were incubated for 2 hours at room temperature. Cells were then washed once with PBS and once with 0.5% BSA in PBS. Thereafter, secondary antibodies (1:100 in 0.05% BSA in PBS) were added and the cells were again incubated for 2 hours at room temperature. The primary and secondary antibodies used are listed in Table 2. The cells were then washed twice with PBS and resuspended in PBS for detection. Three biological repeats were performed and 15 000 cells were analyzed per sample. The efficacy of the assay was investigated by using PrP^{c} as a positive control as LRP/LR binds to it with high affinity and chloramphenical acetyl transferase (CAT) as a negative control as LRP/LR has been shown not to bind to it.

The following samples were prepared:

1. Unstained cells

2. Cells labelled with anti-human PE only

3. Cells labelled with anti-mouse CF™ 647 only

4. Cells labelled with anti-rabbit CF™ 647 only

5. Cells labelled with IgG1-iS18-PE (LRP/LR detection)

6. Cells labelled with anti-PrP^{c}-CF™ 647 (PrP^{c} detection)

7. Cells labelled with anti-CAT-CF™ 647 (CAT detection)

8. Cells labelled with anti-Tau-CF™ 647 (Tau detection)

9. Cells labelled with both IgG1-iS18-PE and anti-PrP^{c}-CF™ 647 (positive control)

10. Cells labelled with both IgG1-iS18-PE and anti-CAT-CF™647 (negative control)

11. Cells labelled with both IgG1-iS18-PE and anti-Tau-CF™ 647

TABLE 2
Antibodies used for Försters Resonance Energy Transfer (FRET)

| Target | | |
|---|---|---|
| Protein | Primary Antibody | Secondary Antibody |
| LRP/LR | Human anti-LRP/LR | Anti-human IgG-PE |
| | (IgG1-iS18) | (Abcam: ab7006) |
| Total Tau | Mouse anti-Tau | Anti-mouse IgG-CF ™ 647 |
| (Tau-5) | (Tau-5) | (Sigma Aldrich: SAB4600182) |
| | (EMD: MAB361) | |
| Prion | Mouse anti-Prion | Anti-mouse IgG-CF ™ 647 |
| Protein (PrP^{c}) | Protein (Sigma | (Sigma Aldrich: SAB4600182) |
| | Aldrich: P0110) | |
| CAT | Rabbit anti-CAT | Anti-rabbit IgG-CF ™ 647 |
| | (Sigma Aldrich: | (Sigma Aldrich: SAB4600184) |
| | C9336) | |

### Stable Overexpression of LRP::FLAG

In order to determine the effect of LRP::FLAG overexpression on tau (τ) levels, cells were transfected with the plasmid, pCIneo-moLRP-FLAG (see Vana and Weiss, A Trans-dominant Negative 37 kDa/67 kDa Laminin Receptor Mutant Impairs PrPSc Propagation in Scrapie-infected Neuronal Cells. J Mol Biol 358: 57-66, 2006, which is incorporated herein by reference thereto). Cells were cultured until 40-50% confluency was achieved. The cells were then transfected using Xfect™ Transfection Reagents (Takara). Briefly. 5 μg of plasmid DNA was mixed with 100 μl Xfect Reaction Buffer and 1.5 μl Xfect Polymer and incubated at room temperature for 10 minutes. This allowed the lipophilic nanoparticle complexes to form around the construct. The mixture was then added to the cells prior to a three-day incubation. Thereafter, a media change was performed and the cells were permitted to proliferate for two days, giving the cells sufficient time to recover from the transfection process and produce the LRP::FLAG protein. The antibiotic Geneticin was then used as a selective treatment. Initially, a high concentration of the antibiotic (6000-8000 ng/μl) was administered until all the non-transfected cells had died. Thereafter, cells were cultured in 2000-4000 ng/μl of Geneticin to ensure the maintenance of LRP::FLAG expression. Stable transfection was achieved after six weeks of Geneticin treatment.

### Western Blotting

### Bicinchoninic Acid (BCA) Assay

BCA assays were performed in order to quantify the total protein concentration present in a cell lysate post protein extraction. This ensured that equal protein concentrations were used for the subsequent sodium dodecyl sulphate polyacrylamide gel electrophoresis (SDS-PAGE) and western blotting procedures. The peptide bonds present in protein cause the Cu²⁺ ions in BCA to reduce to Cu⁺ ions, which results in a colorimetric change that can be measured using an ELISA plate reader.

Cells were harvested as described herein above. Thereafter, they were incubated in cold radioimmunoprecipitation (RIPA) buffer for 10 minutes in order to lyse the cells. The cell lysates were diluted 1:5 with distilled water and 25 μl was loaded into individual wells on a 96-well plate in triplicate. The samples were incubated in 200 μl of 98% BCA and 2% copper (II) sulphate (CuSO₄) at 37° C. for 20 minutes. BSA of known concentrations was used to construct a standard curve. BSA standards were prepared by dilution to produce dilutions ranging from 0 mg/ml to 1 mg/ml, in 0.2 mg/ml increments. Thereafter, 25 μl were loaded in triplicate on the same plate and were incubated in the same mixture. Thereafter, the plate was measured at a wavelength of 562 nm using the Multiskan® GO ELISA plate reader (Thermo Scientific). Total protein concentrations were then extrapolated from the BSA standard curve.

### SDS-PAGE and Western Blotting

SDS-PAGE was used in order to separate the whole protein extract by size, allowing the approximate size of the target protein to be assessed. Thereafter, the proteins were transferred to a membrane via western blotting. Western blotting uses specific antibodies to immunologically detect and quantify the protein levels of the target protein.

The SDS-PAGE procedure was performed using 12% (w/v) Tris-HCl gels allowing the resolving of proteins of 10-200 kDa. All samples were assayed in triplicate. Loading Buffer (40 mM Dithiothreitol) was added to the soluble protein samples and they were heated at 95° C. for 5 minutes prior to loading. A total of 10 μg (β-Actin), 30 μg (LRP::FLAG) and 60 μg (Tau, phospho-Tau (pS404), phospho-Tau (pT231), hTERT, phospho-TERT (pS824) and PrP) was then separated on the gel. A prestained molecular weight marker was loaded onto each gel.

The proteins were separated at 150 V for approximately 1 hour in 1× electrophoresis buffer (25 mM Tris, 192 mM glycine and 0.1% SDS). Thereafter, the separated proteins were transferred onto a polyvinylidene fluoride (PVDF) membrane using a semi-dry transfer apparatus. Transfer was performed at 300 mV for 45-50 minutes using 1× transfer buffer (20% methanol in 25 mM Tris and 192 mM glycine). The proteins were fixed to the membrane in 0.4% formaldehyde for 30 minutes. The membrane was then blocked in 3% BSA in 0.1% PBS-Tween for an hour in order to prevent non-specific antibody binding. The membranes were incubated overnight at 4° C. with primary antibody as per Table 3. The next day, five 0.1% PBS-Tween washes were performed for 5 minutes each to remove any unbound antibody. The membranes were then incubated for an hour at room temperature with the appropriate horse radish peroxidise (HRP)-coupled secondary antibody as per Table 3. Again, the membrane was washed with 0.1% PBS-Tween as above. The membranes were then incubated with Thermo Scientific chemiluminescent substrate, which reacts with the HRP enzyme to form a precipitate that can be detected using radiation exposure. The membranes were then imaged using the Bio-Rad GelDoc XR Imager. In SH-SY5Y cells. LRP::FLAG was detected using mouse anti-FLAG® conjugated to the fluorescent probe, Cy3 (Sigma Aldrich: A-9594) (Table 3.3) as the anti-mouse HRP antibody (Abcam: ab97023) bound non-specifically. Thus, this blot was not treated with chemiluminescent substrate and was instead viewed on the GelDoc using Cy3 fluorescence. The blots were then analyzed using the Bio-Rad Image Lab 4.0 software and densitometry was performed in order to obtain protein levels relative to the loading control. β-Actin.

TABLE 3
Antibodies used for western blotting

| Target | Primary | | Secondary | |
|---|---|---|---|---|
| Protein | Antibody | Dilution | Antibody | Dilution |
| β-Actin | Mouse anti-β- | 1:10000 | — | — |
| | Actin | | | |
| | Peroxidase | | | |
| | (Sigma Aldrich: | | | |
| | A3854) | | | |
| LRP::FLAG | Mouse anti- | 1:6666 | Anti-mouse | 1:10000 |
| | FLAG ® M2 | | IgG-HRP | |
| | (Sigma Aldrich: | | (Abcam: | |
| | F-3165) | | ab97023) | |
| LRP::FLAG | Mouse anti- | 1:5000 | — | — |
| | FLAG ® | | | |
| | M2-Cy3 ™ | | | |
| | (Sigma Aldrich: | | | |
| | A-9594) | | | |
| Total Tau | Mouse anti-Tau | 1:500 | Anti-mouse | 1:3333 |
| (Tau-5) | (Tau-5) (EMD: | | IgG-HRP | |
| | MAB361) | | (Abcam: | |
| | | | ab97023) | |
| Phospho-Tau | Rabbit anti-p-Tau | 1:1000 | Anti-rabbit | 1:3333 |
| (pS404) | (S404) (Cell | | IgG-HRP | |
| | Signalling: | | (Cell | |
| | 20194S) | | Signalling: | |
| | | | 7074S) | |
| PHF-Tau | Mouse anti- | 1:500 | Anti-mouse | 1:3333 |
| (pT231) | Phospho-Tau | | IgG-HRP | |
| | (Thr231) (AT180) | | (Abcam: | |
| | (Invitrogen: | | ab97023) | |
| | MN1040) | | | |
| hTERT | Mouse anti-hTERT | 1:2500 | Anti-mouse | 1:3333 |
| | (Santa Cruz | | IgG-HRP | |
| | Biotechnology: | | (Abcam: | |
| | sc-77511) | | ab97023) | |
| Phospho- | Rabbit anti- | 1:1000 | Anti-rabbit | 1:3333 |
| hTERT | phospho- | | IgG-HRP | |
| (pS824) | Telomerase | | (Cell | |
| | (pSer⁸²⁴) | | Signalling: | |
| | (Sigma Aldrich: | | 7074S) | |
| | SAB4504295) | | | |
| Prion | Mouse anti-Prion | 1:5000 | Anti-mouse | 1:3333 |
| Protein | Protein (Sigma | | IgG-HRP | |
| (PrP^{c}) | Aldrich: P0110) | | (Abcam: | |
| | | | ab97023) | |

### Statistical Analysis

All experiments were performed in triplicate, allowing standard deviation to be calculated and statistical analysis to be performed. All statistical analysis of the data obtained was carried out using Microsoft Excel 2016 (Microsoft Corporation). Analysis of results was performed in order to determine Whether the results collected were significantly relevant. The two-tailed Student's t-test was performed at a 95% confidence interval. Values of *p<0.05 were considered significant, while values of **p<0.01 and ***p<0.001 were considered very and highly significant, respectively.

### Results

LRP/LR Co-Localizes with Tau (τ) and hTERT

In order to determine whether LRP/LR interacts with tan (τ), it was important to investigate the sub-cellular localization of these proteins. Since hTERT interacts with LRP/LR and plays a role in tauopathy, its localization was also examined.

Confocal microscopy was performed to determine whether LRP/LR and tau (τ) co-localize (FIG. 1). The merged panels (FIG. 1: D, J) represent a merge of the red tau (frame A) or hTERT signal with the green LRP/LR signal (frame B), with areas of yellow (frames D and J) representing overlap. The co-localization panels (FIG. 1: E, K) clearly label the areas of overlap in white, making co-localization easier to visualize. Lastly, the 2D cytofluorogram (FIG. 1: F, L) is a diagrammatic representation of the laser signals whereby a diagonal in the third quadrant represents a strong co-localization of the proteins. Therefore, as seen in FIG. 1. LRP/LR and tau (τ) co-localize in the perinuclear compartment of HEK-293 cells. Thus, it is possible that these proteins can associate with each other.

FIG. 1 illustrates the co-localization between LRP/LR (green frame B) and tau (τ) (red frame A). Frame A) Endogenous tan levels in HEK-293 cells. Tau (T) localizes to the cytosol. Frame B) Endogenous LRP/LR levels in HEK-293 cells. LRP/LR localizes to the cytosol, nucleus and cell surface. Frames C. I) Nuclei are stained with DAPI (blue). Co-localization occurs between LRP/LR and tau (τ) in the perinuclear compartment, represented by yellow fluorescence in the merged Frame (D), as white areas Frame (E) and as fluorescence in the third quadrant of the 2D cytofluorogram Frame (F). G) CF 647™ only control. Frame H) FITC only control. The merged image Frame (J), co-localization image Frame (K) and 2D cytofluorogram Frame (L) show that there is no co-localization between the negative controls. All images were taken with the Zeiss LSM 780 microscope with the addition of Airyscan™ at a 630× magnification. Scale bars represent 20 μm.

### LRP/LR and Tau (1) Interact Directly

Since LRP/LR was shown to co-localize with tau (τ), it was investigated whether the proteins interact directly with each other by detecting if FRET occurred between the two immunolabelled proteins. As stated previously, a non-radiative transfer of energy, known as FRET, will occur between fluorochromes if they are within 1-10 nm of each other (Förster, 1948), thus allowing protein interactions to be assessed.

Flow cytometry was employed in order to examine FRET in HEK-293 cells. The FL3 filter of the Accuri C6 Flow Cytometer (BD Biosciences) was used to determine the presence of FRET between the proteins of interest (FIG. 2). Within this channel, the acceptor. CF™ 647, does not exhibit fluorescence, but may be indirectly excited by FRET if it is in close proximity to the donor, PE. Thus, a shift in fluorescence in the FL3 channel is indicative of an interaction between the proteins of interest.

Therefore, the shift observed in CF™ 647 fluorescence intensity when cells were co-labelled with LRP/LR-PE and Tau-CF™ 647 (FIG. 2: F) shows that FRET has occurred between these two proteins. Thus. LRP/LR and tau (τ) are within 10 nm of each other and it can be concluded with high probability that the proteins interact. In this experiment, PrP^{c} was used as a positive control as LRP/LR binds to it with high affinity, and CAT was used as a negative control as LRP/LR has been shown not to bind to it. The CF™ 647 fluorochrome is not excited by the 488 mu laser and did not exhibit a fluorescence emission in the FL3 channel as the fluorescence intensity of the proteins labelled with the antibody (PrP^{c}, CAT and Tau) was able to be superimposed on that of the unstained cells (FIG. 2: A, C, E). Upon co-labelling of cells with LRP/LR-PE and PrP^{c}-CF™ 647 (FIG. 2: B), there was shift in the CF™ 647 histogram along the FL3 fluorescence intensity axis, which is indicative of the interaction between these proteins. Conversely, upon co-labelling with LRP/LR-PE and CAT-CF™ 647 (FIG. 2: D), there was no shift in the CF™ 647 histogram, thus no effect on the emission of CF™ 647 was observed.

FIG. 2 shows flow cytometric analysis of FRET between intracellular LRP/LR and tau (τ). The fluorescence intensity of the unstained HEK-293 cells (dark blue histogram—i) was superimposed with that of cells labelled with the CF™ 647 secondary antibody only (yellow histogram—ii) as well as with cells in which the proteins of interest; PrP^{c}, CAT and tau, were labelled with CF™ 647 (A. C. E). B) Co-labelling of LRP/LR-PE and PrP^{c}-CF™ 647 (positive control)(pink histogram—iii). D) Co-labelling of LRP/LR-PE and CAT-CF™ 647 (negative control) (green histogram—iv). F) Co-labelling of LRP/LR-PE and Tau-CF™ 647 (red histogram—v). Each panel is a representative image. Three biological replicates were conducted.

### Confirmation of LRP::FLAG Overexpression in HEK-293 and SH-SY5Y Cells

HEK-293 and SH-SY5Y cells were stably transfected with the pCIneo-moLRP-FLAG plasmid. This was done in order to increase total LRP levels in the cells by overexpressing LRP::FLAG. For clarification purposes, cells transfected with the pCIneo-moLRP-FLAG plasmid will be identified as transfected cells, while those that have not been transfected with the plasmid will be identified as non-transfected cells.

Western blotting was used to determine whether the cells had been successfully transfected with the plasmid (FIG. 3). The detection of the LRP::FLAG protein in the transfected cells shows that both the HEK-293 (FIG. 3: A) and SH-SY5Y (FIG. 3: B) cells were stably transfected.

### Overexpression of LRP::FLAG Decreases Total Tau (τ) and Phospho-Tau Levels

HEK-293 and SH-SY5Y cells were stably transfected with the pCIneo-moLRP-FLAG plasmid to induce an overexpression of LRP::FLAG. Western blotting confirmed the transfection of both HEK-293 and SH-SY5Y cells (FIG. 2A: C). After confirming transfection of both cell lines, western blotting was utilized to determine the effect of LRP::FLAG overexpression on both total and phosphorylated tau levels (FIG. 2A: C). A significant decrease in total tau levels of 45% and 35% was observed in transfected HEK-293 (FIG. 2B) and transfected SH-SY5Y cells (FIG. 2D), respectively. Since it is not possible to investigate hyperphosphorylation of tau in vitro, two antibodies were used which target tau at two different phosphorylation sites (pS404 and pT231). In transfected HEK-293 cells there was a significant 68% reduction in tau levels phosphorylated at 5404 and a significant 98% reduction in tau levels phosphorylated at T231 (FIG. 2B). In transfected SH-SY5Y cells, a significant 63% (phospho-Tau pS404) and 92% (phospho-Tau pT231) reduction in protein levels was observed, respectively (FIG. 2D). Since these are two of the major culprits of tauopathy, the decreased phosphorylation at these two sites together may indicate a possible decrease in hyperphosphorylated tau. This decrease in phosphorylated tau was also observed in the cytoplasm and the nucleus of both HEK-293 and SH-SY5Y cells by using confocal microscopy. Therefore the overexpression of LRP::FLAG might decrease the overall hyperphosphorylation of tau.

Confocal microscopy was further employed to analyze the expression of tau (τ) and phospho-tau (τ) intracellularly as these proteins are normally found in the cytoplasm and nucleus (FIG. 5). The merged panels represent the merge of the red staining, representing tau or phospho-tau, with the blue nuclear DAPI staining. These results confirm the reduction of total tau and phospho-tau in all cellular locations in transfected cells relative to the non-transfected cells.

FIG. 5 shows intracellular expression of tau and phospho-Tau in transfected and non-transfected HEK-293 and SH-SY5Y cells. b, e) Nuclei are stained with DAPI (blue). A, B) Expression of total tau decreases in transfected HEK-293 and transfected SH-SY5Y cells. Endogenous levels of total tau (red) (a). Levels of total tau (red) after transfection with pCIneo-moLRP-FLAG plasmid (d). Merge of total tau with DAPI staining (c, f). C. D) Expression of phospho-Tau (pS404) is decreased in transfected HEK-293 and transfected SH-SY5Y cells. Endogenous levels of phospho-Tau (pS404) (red) (a). Levels of phospho-Tau (pS404) (red) after transfection with pCIneo-moLRP-FLAG plasmid (d). Merge of phospho-Tau (pS404) with DAPI staining (c, f). E. F) Expression of phospho-Tau (pT231) is decreased in transfected HEK-293 and transfected SH-SY5Y cells. Endogenous levels of phospho-Tau (pT231) (red) (a). Levels of phospho-Tau (pT231) (red) after transfection with pCIneo-moLRP-FLAG plasmid (d). Merge of phospho-Tau (pT231) with DAPI staining (c, f). All images were taken at 630× magnification with the addition of Airyscan™. Scale bars represent 20 μm.

FIG. 9 shows intracellular expression of tau and phospho-Tau in transfected and non-transfected HEK-293 and SH-SY5Y cells. b, e) Nuclei are stained with DAPI (blue). A, B) Expression of total tau decreases in transfected HEK-293 and transfected SH-SY5Y cells. Endogenous levels of total tau (red) (a). Levels of total tau (red) after transfection with pCIneo-moLRP-FLAG plasmid (d). Merge of total tau with DAPI staining (c, f). C. D) Expression of phospho-Tau (pS404) is decreased in transfected HEK-293 and transfected SH-SY5Y cells. Endogenous levels of phospho-Tau (pS404)(red) (a). Levels of phospho-Tau (pS404) (red) after transfection with pCIneo-moLRP-FLAG plasmid (d). Merge of phospho-Tau (pS404) with DAPI staining (c, f). E, F) Expression of phospho-Tau (pT231) is decreased in transfected HEK-293 and transfected SH-SY5Y cells. Endogenous levels of phospho-Tau (pT231) (red) (a). Levels of phospho-Tau (pT231) (red) after transfection with pCIneo-moLRP-FLAG plasmid (d). Merge of phospho-Tau (pT231) with DAPI staining (c, f).

Overexpression of LRP::FLAG Increases Total hTERT Levels

LRP/LR has also recently been found to interact with the anti-ageing related proteins telomerase and hTERT [36]. In AD, neuronal cells have critically shortened telomeres as an accumulation of Aβ which is able to inhibit the activity of telomerase. Furthermore, there is an absence of hTERT in cells affected by hyperphosphorylated tau. Since both LRP/R and telomerase are known to play a role in the Aβ facet of AD, it was hypothesized that they could also play a role in tauopathy. It is known that overexpression of LRP::FLAG increases hTERT levels in HEK-293 cells and SH-SY5Y cells. It has also been shown that this increase results in an increase in telomerase activity [36,58]. Thus, western blotting was performed to examine the effect of LRP::FLAG overexpression on hTERT levels, together with the active form of hTERT, phospho-TERT (pS824) (FIG. 6A; C), as this is indicative of an increase in telomerase activity. A significant increase of 120% and 125% in hTERT levels was observed in transfected HEK-293 (FIG. 6B) and SH-SY5Y (FIG. 6D) cells, respectively. A significant increase in phospho-TERT levels was also observed in both cell lines, with a 112% increase in transfected HEK-293 cells (FIG. 6B) and a 96% increase in transfected SH-SH5Y cells (FIG. 6D).

TABLE 4
Ratio of phospho-TERT to hTERT in transfected SH-SY5Y cells.
Analysis was performed relative to hTERT levels which were set to 1.
Transfected SH-SY5Y Cells

| | Ratio | p-value |
|---|---|---|
| phospho-TERT: hTERT | 0.516 ± 0.037 | 2.20571E−05 (***) |

n = 3 biological repeats.
*p: 0.05,
**p: 0.01,
(***) p: 0.001

### Overexpression of LRP::FLAG Decreases Aβ Expression and PrP^{c} Levels

Aβ₂ expression was investigated using confocal microscopy.

An overall decrease in Aβ₄₂ expression was observed in transfected HEK-293 cells compared to non-transfected cells (FIG. 7). The merged panels represent the merge of the green staining, representing Aβ₄₂, with the blue DAPI nuclear staining. Therefore, the decrease in Aβ₄₂ expression may contribute to the decreases in phosphorylated tau (τ).

FIG. 7 shows intracellular expression of Aβ₄₂ in transfected and non-transfected HEK-293 cells. Expression of Aβ₄₂ decreases in transfected HEK-293 cells. A) Endogenous levels of Aβ₄₂. Aβ₄₂ localizes to the cytosol and cell surface. D) Levels of Aβ₄₂ after transfection with pCIneo-moLRP-FLAG plasmid. B, E) Nuclei are stained with DAPI. C, F) Merge of Aβ₄₂ with DAPI staining. All images were taken with the Zeiss LSM 780 microscope with the addition of Airyscan™ at a 630× magnification. Scale bars represent 20 μm.

The effect of LRP::FLAG overexpression on PrP^{c} was investigated using western blotting (FIG. 8: A1 and B1).

Endogenous PrP^{c} can exist as three isoforms: non-glycosylated, monoglycosylated and diglycosylated and thus ranges in size from approximately 20 kDa to approximately 35 kDa. In HEK-293 cells, both the di- and monoglycosylated forms were seen (FIG. 8: A1), while in SH-SY5Y cells only the monoglycosylated form was observed (FIG. 8: B1). It is important to note that glycosylation is not necessary for encoding strain information in prions and that PrP^{c} glycoform ratios differ across brain regions and across cell lines. Therefore, the differences in total were PrP^{c} examined.

A significant decrease in total PrP^{c} in both transfected HEK-293 and SH-SY5Y cells of 34% (FIG. 8: A2) and 39% (FIG. 8: B2), respectively was observed. This decrease may be important in the signal transduction pathway between Aβ₄₂ and PrP^{c} which could result in a decreased in tau hyperphosphorylation.

FIG. 8 shows overexpression of LRP::FLAG in HEK-293 and SH-SY5Y cells decreases PrP^{c} levels. A1, B1) Lanes 1-3: non-transfected cells. Lanes 4-6: transfected cells. B-Actin (HRP) was used as a loading control. Densitometric analysis performed was relative to the non-transfected HEK-293 (A2) and SH-SY5Y (B2) cells which were set to 100%. Error bars represent standard deviation, n=3 biological repeats. *p: 0.05. **p: 0.01, ***p: 0.001; Student's t-test.

### Further Comments on the Experimental Results:

Studies targeting LRP/LR downregulation have successfully impeded Aβ shedding and rescued cells from Aβ-induced cytotoxicity. These observations have also been confirmed in vivo, whereby Alzheimer's Disease (AD) transgenic mice treated with the anti-LRP/LR antibody, IgG1-iS18, which blocks cell surface LRP % LR, showed decreased amyloid plaque generation with a corresponding improvement in short-term and learning memory.

However, it was not known whether LRP/LR upregulation has an effect on the other neuropathological agent of AD, hyperphosphorylated tau. Herein it is demonstrated that LRP/LR interacts directly with tau and that overexpression of LRP::FLAG decreases total and phosphorylated tau levels. This was not expected. In addition. LRP::FLAG overexpression increases hTERT and phospho-TERT levels with a concomitant decrease in PrP^{c} levels.

Initially, confocal microscopy was used to determine whether LRP/LR and tau co-localize in HEK-293 cells, as this is a pre-requisite for a possible interaction between the proteins. Co-localization between LRP/LR and tau in the nuclear and cytosolic regions was observed as shown in the figures. Thereafter. FRET was performed to determine if these proteins interact directly. The fluorescence shift observed in indicates that these proteins exist within 10 nm of each other. Therefore, our results show that LRP/LR and tau directly interact with each other. LRP/LR is involved in the main function of tau: to assemble and maintain microtubules in healthy cells.

In order to investigate whether LRP/LR plays a role in tauopathy. HEK-293 and SH-SY5Y cells were stably transfected with the pCIneo-moLRP-FLAG construct in order to induce overexpression of LRP::FLAG. The effect of this overexpression on both total and phosphorylated tau levels was determined using western blotting. A significant decrease in phosphorylated tau (pS404 and pT231) was observed in both cell lines. The decreased phosphorylation observed at these two sites may indicates decrease in hyperphosphorylated tau. Confocal microscopy confirmed that this decrease in phosphorylated tau was observed in the cytoplasm and the nucleus of both cell lines. Hyperphosphorylation of tau is a major problem in AD as it forms tangles that disrupt the internal structure of neuronal cells by de-stabilizing the cytoskeletal tracks. Thus, the significant decrease observed in both tau phosphorylation sites (pS404 and pT231) results in a reduced amount of aggregation of hyperphosphorylated tau, resulting in less damage to neuronal cells. Since this aggregation is known to cause disintegration of cytoskeletal tracks, a decrease in hyperphosphorylated tau hinders cytoskeleton disintegration and improve vital cell processes, such as axonal transport, thereby permitting neurons to retain normal functioning in AD patients. In particular, the phosphorylation of tau at S404 and T231, which was investigated herein, is known to negatively affect the binding of the tau protein to the microtubules, thereby de-stabilizing them and inhibiting cytoskeletal signalling and transport.

T231 mutant N18 neuroblastoma cells abolished phosphorylation of tau by the serine/threonine kinase GSK3, indicating that phosphorylation at T231 plays an important role in the hyperphosphorylation of tau. Therefore, a decrease in phosphorylation at these sites prevents the dissociation of tau and promote tubulin polymerization and assembly into microtubules. Thus, the structural integrity of the cells would be maintained, and their signalling and transport capabilities would remain intact, allowing them to better resist the damaging effects of AD. In particular, DNA damage repair proteins can be trafficked to the sites of cytotoxicity in order to rescue the cells. The DNA damage caused by the cytotoxicity present in AD results in neuronal apoptosis via oxidative stress.

In addition, hyperphosphorylation of tau is able to enhance Aβ cytotoxicity by sequestering Fyn kinase. Fyn is thus re-distributed to the neuron whereby it can strengthen the excitotoxic signalling of the neurotransmitter glutamate, which is known to enhance the cytotoxicity of Aβ oligomers. Therefore, a decrease in phosphorylated tau and the resultant decrease in neurofibrillary tangles could both lessen cytotoxicity as well as allow DNA repair to occur. Overall, the decrease in phosphorylated tau observed could be acting as a mitigator for AD development and progression. Combination therapy as provided herein may ameliorate AD effectively.

Furthermore, a decrease in total tau levels was observed in both analyzed cell lines. Wild-type tau is essential for the functioning of microtubules and cell signalling, therefore a decrease in total tau may be detrimental to the cells. However, it was seen that LRP::FLAG overexpression rescues cells from cytotoxicity after treatment with Aβ₄₂. While overexpression of LRP::FLAG does not specifically target phosphorylated tau, it seems to have a much more notable effect on the phosphorylated form. LRP-laminin interaction could also affect the phosphorylation of tau, especially as LRP/LR was shown to directly interact with tau. Furthermore. LRP/LR and focal adhesion kinase (FAK) have been shown to interact after LRP/LR binds to laminin. Indeed, in neurons treated with Aβ there is also increased phosphorylation of FAK and an increased association between FAK and Fyn kinase in addition to the increased phosphorylation of tau. Taken together, these increases are known to enhance cytotoxicity in AD.

Therefore, it is possible that the interaction between LRP and FAK, and the subsequent activation of signalling pathways, such as PI3-kinase/Akt and MAPK, affect the phosphorylation of tau in AD.

The effect of LRP::FLAG overexpression on hTERT levels, together with the active form of hTERT, phospho-TERT (pS824), is indicative of an increase in telomerase activity. Significant increases in both hTERT and phospho-TERT (pS824) levels were observed in both cell lines. An increase in hTERT induces/causes a corresponding increase in telomerase activity. However, in order to be active, hTERT has to be present in the nucleus. Therefore, in order for nuclear localisation of the full length 120 kDa hTERT variant to occur, it firstly undergoes phosphorylation at the serine residues 227 and/or 824 by PI3-kinase/AKT signalling. This suggests that the increase in telomerase activity is as a result of not only increased hTERT levels but also hTERT import to the nucleus.

Indeed, by comparing phospho-TERT and hTERT levels in HEK-293 and SH-SY5Y cells, it is clear that not all hTERT is being converted into phospho-TERT. This hTERT may thus be involved in extra-telomeric functions. Of note is the protective function of hTERT against ROS, hTERT is able to migrate to the mitochondria when cells are under oxidative stress, which allows hTERT to interact with mtDNA in order to convey protection. In AD, there is compromised energy production in the brain due to mitochondrial dysfunction. This dysfunction can increase ROS production which has been shown to promote the hyperphosphorylation of tau, although it has not yet been determined how this occurs. Conversely, hyperphosphorylation of tau and the formation of neurofibrillary tangles could also impair the movement of mitochondria along microtubules. In neurons, mitochondria are distributed to areas of the axon where metabolic demand is high, such as synapses, and active growth cones or branches. As a result. ATP depletion can occur in other areas of axons and dendrites which can worsen synaptic dysfunction. Importantly, in AD, the absence of hTERT has been shown to increase the levels of mitochondrial superoxide in neurons and truncated tau expressed in vivo generates increased oxidative stress over time through an accumulation of ROS. Furthermore, pathological tau and hTERT have been observed to be mutually exclusive, suggesting that neurons expressing hTERT may be prevented from developing tau pathology. LRP::FLAG overexpression causes an increase in hTERT in the cytoplasm and nucleus and it is this increase in the cytoplasm and nucleus which could correspond to an increase in the extra-telomeric functions of hTERT. Therefore, in AD, the increase in hTERT observed, particularly in the cytoplasm, improves mitochondrial function and protects against ROS production. Furthermore, nuclear hTERT is able to regulate inflammatory and proliferative pathways, such as Wnt and NF-κB. Therefore, the increased nuclear hTERT observed will regulate pro-proliferative and anti-inflammatory genes via these pathways which could prevent apoptosis and aid cell survival in AD. Therefore, an increase in hTERT may prevent cell cycle arrest and apoptosis in a number of ways: through increased telomerase activity and telomere elongation, by reducing ROS production thereby improving mitochondrial function, as well as by regulating cell survival pathways. Overall, an increase in hTERT slows the progression of tauopathy and AD.

A decrease in both total PrP^{c} and phosphorylated tau was observed in cells overexpressing LRP::FLAG. The decrease in PrP^{c} observed may be beneficial in preventing the Aβ-PrP^{c} signalling cascade occurring in the later stages of AD. Therefore LRP::FLAG overexpression is able to decrease phosphorylated tau. Aβ₄₂ and PrP^{c} levels. These proteins interact with each other during progression of AD, whereby Aβ₄₂ binds to PrP^{c} causing cytotoxicity and increased Aβ₄₂ shedding, which results in hyperphosphorylation of tau and the formation of neurofibrillary tangles. It is also important to note that LRP::FLAG does not have an effect on PrP^{Sc} levels, the causative agent for prion disorders such as Creutzfeldt-Jakob disease.

LRP/LR and tau share the same sub-cellular locations and directly interact with each other. Therefore, cytoplasmic LRP could assist with the stabilization of microtubules through their binding to tau peptides. LRP::FLAG overexpression caused a decrease in phosphorylated tau and PrP^{c} levels. Taken together the overexpression of LRP::FLAG is able to affect decrease the cytotoxic effects observed in AD. Furthermore, hTERT levels increased after LRP::FLAG overexpression and thus could provide protection against the cytotoxicity caused by tau hyperphosphorylation. LRP::FLAG acts as a potential alternative therapeutic tool for AD treatment through a rescuing mechanism of AB-mediated cytotoxicity and a decrease of phosphorylated tau levels which might be mediated by increased hTERT and decreased PrP^{c} levels, respectively. Therefore, elevation of LRP levels is a potential therapeutic strategy for the treatment of AD and other tauopathies.

FIG. 9 shows a proposed mechanisms of LRP::FLAG overexpression in AD cell culture models. A) Direct interaction between LRP and tau: an increase in cytoplasmic LRP::FLAG could stabilize microtubules through the direct interaction of LRP and tau. B) Aβ₄₂-PrP^{c}-hyperphosphorylated tau cascade: LRP::FLAG overexpression decreases the levels of Aβ₄₂ and PrP^{c} which may contribute to decreased phosphorylated tau. C) Increased hTERT: LRP::FLAG overexpression increases hTERT levels which conveys neuroprotection as it is able to protect against oxidative stress. Increased hTERT levels may also decrease the level of intercellular Aβ₄₂ and phosphorylated tau, however, the exact mechanism is unknown. LRP::FLAG overexpression also results in decreased senescent marker proteins γH2AX and β-galactosidase. Dotted lines and question marks indicate an unknown mechanism.

### Ongoing Experimental

in vivo studies are ongoing. Tau (t) transgenic mice, such as the P301S strain, are examined regarding the effect of elevating LRP in vivo. These mice express mutant human microtubule-associated protein tau (MAPT) causing them to develop neurofibrillary tangle-like inclusions in the neocortex, amygdale, hippocampus, brain stem and spinal cord. Therefore, they exhibit neuronal loss and brain atrophy by eight months. LRP::FLAG could be injected as a plasmid intranasally, to ensure efficient delivery to the brain. Alternatively, the LRP::FLAG sequence could be inserted into a viral vector for delivery, preferably via oral means.

Furthermore, both in vitro and in vivo studies have shown that the anti-LRP/LR specific antibody, IgG1-iS18, can impede neurodegeneration by reducing Aβ levels in AD. Combinatorial treatment using both LRP::FLAG overexpression and IgG1-iS18 blockade of LRP/LR together with upregulation as taught herein is provided. This treatment would target more than one aspect of AD, and thereby improve the outcome of the treatment.

The LRP/LR or plasmid according to the first to further aspects herein could be encapsulated within a lipophilic complex or nanoparticle that could both protect the drug and allow it to cross the plasma membrane of cells. Additionally, brain-specific ligands can be attached to the active molecule to ensure that the drug does not travel anywhere else in the body.

While the subject matter of the disclosure has been described in detail with respect to specific embodiments and/or examples thereof, it will be appreciated that those skilled in the art, upon attaining an understanding of the foregoing may readily conceive of alterations to, variations of and equivalents to these embodiments. Accordingly, the scope of the present disclosure should be assessed as that of the claims and any equivalents thereto, which claims are appended hereto.

1. A method of decreasing concentration of tau (τ) protein and/or phosphorylated tau (τ) protein in a target cell of a human or animal subject having Alzheimer's Disease (AD), the method comprising the following steps:
(i) transfecting the cell to produce 37 kDa/67 kDa laminin receptor precursor/high affinity laminin receptor (LRP/LR) and/or a fragment thereof by employing a transfecting agent; or
(ii) providing the cell with LRP/LR and/or fragments thereof,
such that in use, Steps (i) or (ii) provide an overexpression of LRP/LR in the target cell together with a concomitant increase in reverse transcriptase of telomerase (hTERT), which in turn results in decreasing concentration of tau (τ) protein and/or phosphorylated tau (τ) protein in the target cell, and wherein the target cells are brain cells of a human or animal having AD.

2. The method of claim 1, wherein the transfecting agent and/or the LRP/LR and/or the fragment of LRP/LR are provided as a delivery means formulated as a pharmaceutical composition, which pharmaceutical composition includes a pharmaceutical carrier for parenteral or non-parenteral administration to the subject,
wherein non-parenteral administration is at least one the following group consisting of: oral, nasal, rectal, vaginal, optical and transdermal administration, and
wherein parenteral administration is at least one of the following group consisting of: intravenous, subcutaneous and intramuscular administration.

3. The method of claim 1, wherein the transfecting agent is pCIneo-moLRP::FLAG plasmid and/or the LRP/LR comprises a peptide/protein sequence listing as set forth in SEQ ID NO: 1 or SEQ ID NO: 2, or a fragment thereof as set for in SEQ ID NO: 4 and SEQ ID NO: 5.

4. The method of claim 1, wherein the transfecting agent is pCIneo-moLRP::FLAG plasmid and/or the LRP/LR comprises a peptide/protein sequence listing having at least 80% homology to the sequences as set forth in SEQ ID NO: 1 or SEQ ID NO: 2, or a fragment thereof as set for in SEQ ID NO: 4 and SEQ ID NO: 5.

5. The method of claim 1, further including a Step (iii), wherein Step (in) includes:
(iii) repeating Steps (i) and/or Step (ii) for a first period of time in human or animal subject having Alzheimer's Disease (AD), wherein the first period of time is selected from the group: daily, weekly, or monthly.

6. The method of claim 5, further including a Step (iv), wherein Step (iv) includes:
(iv). down regulation of LPR/LR prior to any one of Steps (i), (ii) and (iii) being completed, alternatively after any of Steps (i), (ii) and (iii) being completed, further alternatively together concomitantly with any one of Steps (i), (ii), and (iii).

7. The method of claim 6, wherein downregulation of LPR/LR includes providing the human or animal subject anti-37 kDa/67 kDa laminin receptor (LRP/LR) specific antibody (or fragment thereof) such that binding occurs between a surface epitope of 37 kDa/67 Kda laminin receptor (LRP/LR) and the anti-37 kDa/67 kDa laminin receptor (LRP/LR) specific antibody (or fragment thereof) causing a decrease in the concentration of amyloid beta (Aβ) peptide in the human or animal.

8. The method of claim 7, wherein downregulating LRP/LR includes providing to the human or animal subject a nucleotide sequence, preferably shRNA of SEQ ID NO: 6 and/or SEQ ID NO: 7, such that binding occurs between mRNA of 37 kDa/67 kDa laminin receptor (LRP/IR) and the shRNA causing downregulation of the 37 kDa/67 kDa laminin receptor (LRP/LR) which in turn causes a decrease in the concentration of amyloid beta (Aβ) peptide in the human or animal.

9. The method of claim 6, wherein the down regulating LPR/LR includes providing the human or animal subject anti-37 kDa/67 kDa laminin receptor (LRP/LR) specific antibody (or fragment thereof) and providing the human or animal subject a nucleotide sequence, preferably shRNA of SEQ ID NO: 6 and/or SEQ ID NO: 7.

10. A biopharmaceutical agent including (i) a 37 kDa/67 kDa laminin receptor precursor/high affinity laminin receptor (LRP/LR) and/or a fragment thereof and/or (ii) a transfecting agent for expressing a 37 kDa/67 kDa laminin receptor precursor/high affinity laminin receptor (LRP/LR) and/or a fragment thereof, for use in decreasing concentration of tau (τ) protein and/or phosphorylated tau (τ) protein in a target cell of a human or animal subject having Alzheimer's Disease (AD), wherein LRP/LR and/or the fragment thereof and/or wherein the transfecting agent being for administration to a target cell of a subject in need thereof, wherein the target cells include brain cells of a human or animal having AD.

11. The biopharmaceutical agent of claim 10, wherein the (i) the 37 kDa/67 kDa laminin receptor precursor/high affinity laminin receptor (LRP/LR) and/or a fragment thereof and/or (ii) the transfecting agent for expressing a 37 kDa/67 kDa laminin receptor precursor/high affinity laminin receptor (LRP/LR) and/or a fragment thereof, are encapsulated into the delivery means, and wherein encapsulation takes place using nanoparticles.

12. The biopharmaceutical agent of claim 11, wherein the nanoparticles are polymeric nanoparticles selected from the following group consisting of: eudragit, gum arabic, carrgeenan, cellulose, hydroxypropyl cellulose (HPC), methylcellulose (MC), hydroxypropylmethylcellulose (HPMC), polylactic-co-glycolic acid (PLGA), chitin, pectin, amylopectic, natural rubber, polyethylene, polypropylene, polystyrene, polyamide, polyacrylonitrile, polyvinyl chloride, polyvinyl alcohol (PVA), polyethylene glycol (PEG), polyethylene oxide (PEO), poly(D-lactide) (PDLA), polylactic acid (PLLA), polygalacturonate, methylcellulose (polyacetals), poly(ε-caprolactone), phospholipids, polysaccharides, polyanionic polysaccharides, carboxymethyl cellulose, carboxymethyl amylose, chondroitin-6-sulfate, dermatin sulfate, heparin, heparin sulfate, poly(hydroxyethyl methylacrylate), collagen, fibrinogen, albumin, fibrin, acrylamide, hydroxypropyl methacrylamide-based copolymers, polyacrylamide, poly(N-isopropyl acrylamide) (pNIPAAm), polyvinylpyrrolidone, poly(methacrylic acid-g-ethylene glycol), poly(N-2-hydroxypropyl methacrylamide), poly(glycolic acid) (PGA), poly(lactic acid) (PLA), chitosan, poly(2-hydroxyethylmethacrylate) (HEMA), polyphazene, phosphorylcholine, hyaluronic acid (HA), hydroxyethyl methacrylate (HEMA), methylene-bis-acrylamide (MBAAm), poly(acrylic acid) (PAAc), poly-acrylamide (PAAm), polyacrylonitrile (PAN), polybutylene oxide (PBO), polycaprolactone (PCL), poly(ethylene imine) (PEI), poly(ethyl methacrylate) (PEMA), propylene fumarate (PF), poly(glucosylethyl methacrylate) (PGEMA), poly(hydroxy butyrate) (PHB), poly(hydroxyethyl methacrylate) (PHEMA), poly(hydroxypropyl methacrylamide) (PHPMA), poly(methyl methacrylate) (PMMA), poly(N-vinyl pyrrolidone) (PNVP), poly(propylene oxide) (PPO), poly(vinyl acetate) (PVAc), poly(vinyl amine), chondroitin sulfate, dextran sulfate, polylysine, gelatin, carboxymethyl chitin, dextran, agarose, pullulan, polyesters, PEG-PLA-PEG, PEG-PLGA-PEG, PEG-PCL-PEG, PLA-PEG-PLA, poly(PF-co-EG), poly(PEG/PBO-terephthalate), PEG-bis-(PLA-acrylate), PEG6CDs, PEG-g-poly(AAm-co-vinlyamine), poly(NIPAAm-co-AAc), poly(NIPAAm-co-EMA), PNVP, poly(MMA-co-HEMA), poly(AN-co-allyl sulfonate), poly(biscarboxy-phenoxy-phosphazene), poly(GEMA-sulfate), poly(PEG-co-peptides), alginate-g-(PEO-PPO-PEO), poly(PLGA-co-serine), collagen-acrylate, alginate, alginate-acrylate, poly(HPMA-g-peptide), HA-g-NIPAAm, and poly(vinyl methyl ether) (PVME), and/or derivatives of any one or more of the aforementioned.

13. The biopharmaceutical agent of claim 12, wherein the nanoparticles are lyophilized.

14. The biopharmaceutical agent of claim 13, wherein the lyophilized nanoparticles are lyophilized poly(lactic-co-glycolic acid)(PLGA) nanoparticles.

15. The biopharmaceutical agent of any one of claims 10 to 14, wherein the delivery means is dispersed within or surrounded by a pharmaceutical carrier to provide a pharmaceutical composition.

16. The biopharmaceutical agent of claim 15, wherein the delivery means and/or the pharmaceutical carrier are functionalized to facilitate site specific delivery to the target cells.

17. The biopharmaceutical agent of any one of claims 10 to 16, further including an active pharmaceutical ingredient (API) selected from the group consisting of: donepezil, rivastigmine, galantamine, memantine and combinations thereof.

18. The biopharmaceutical agent of any one of claims 10 to 17, further including an anti-oxidant such that in use at the target cell the anti-oxidant scavenges reactive oxygen species.

19. The biopharmaceutical agent of any one of claims 10 to 18, further comprising an inhibitor of cytochrome P450 3A4 (CYP3A4), wherein the inhibitor of cytochrome P450 3A4 (CYP3A4) is selected from the group consisting of: polyethylene glycol, polyamine, polymethyl methacrylate and derivatives thereof.

20. The biopharmaceutical agent of claim 19, further comprising a P-glycoprotein (P-gp) efflux pump inhibitor.

21. The biopharmaceutical agent of claim 20, further comprising an enteric coating thereabout and formulated for oral delivery as a tablet.

22. A method of treating and/or preventing Alzheimer's Disease (AD), the method including the following steps:
(i). transfecting the cell to produce 37 kDa/67 kDa laminin receptor precursor/high affinity laminin receptor (LRP/LR) and/or a fragment thereof; or
(ii). providing the cell with LRP/LR and/or fragments thereof,
such that in use, Steps (i) or (ii) provide an overexpression of LRP/LR in the target cell together with a concomitant increase in reverse transcriptase of telomerase (hTERT), which in turn results in decreasing concentration of tau (τ) protein and/or phosphorylated tau (τ) protein in a target of a human or animal having Alzheimer's Disease, and wherein the target cells are brain cells of a human or animal having AD.
